(19) United States
(12) Patent Application Publication
(10) Pub. No.: US 2022/0401689 A1
(43) Pub. Date: Dec. 22, 2022

(54) SYSTEMS AND METHODS FOR ENHANCING SLEEP PATTERNS

(71) Applicant: Delos Living LLC, New York, NY (US)

(72) Inventors: Carolina CAMPANELLA, New York, NY (US); Carolyn SWOPE, New York, NY (US); Chuan HE, Bellevue, WA (US); Yiwen DI, Shanghai (CN); Chenyang LU, New York, NY (US); Jie ZHAO, Hoboken, NJ (US)

(21) Appl. No.: 17/762,835

(22) PCT Filed: Sep. 22, 2020
(86) PCT No.: PCT/US20/52016

§ 371 Date: Mar. 23, 2022

Related U.S. Application Data
(60) Provisional application No. 62/904,952, filed on Sep. 24, 2019.
(60) Provisional application No. 62/966,369, filed on Jan. 27, 2020.

(51) Int. Cl. A61M 21/00

(52) U.S. Cl. CPC A61M 21/00; A61M 2021/0083; A61M 2230/005; A61M 2205/3303; A61M 2205/502; A61M 2205/27; A61M 2205/3561; A61M 2021/005

(57) ABSTRACT

Systems and methods for enhancing wellness in a habitable space are provided. In some forms, an example system for enhancing wellness in a habitable space includes a control system having a processor, communication circuitry, and a memory. The communication circuitry of the control system is configured to communicate with one or more devices positioned within the habitable space. One or more sensors may also be positioned proximate the habitable space to detect indicia (e.g., biometric characteristics of a user, behavioral characteristics of a user, and environmental characteristics) and communicate the detected indicia to the control system. Based at least in part on the detected indicia, the control system is configured to trigger initiation of a scene in the habitable space by communicating a signal to at least one of the devices in the habitable space to adjust operation thereof.

### CROSS-REFERENCE TO RELATED APPLICATIONS

This application claims the benefit of U.S. Provisional Application No. 62/966,369, filed Jan. 27, 2020, and U.S. Provisional Application No. 62/904,952, filed Sep. 24, 2019, which are incorporated herein by reference in their entirety.

### FIELD

This application generally relates to habitable spaces, and more particularly, techniques for enhancing sleep patterns in such spaces.

### BACKGROUND

Sleep can be an important contributor to a person's well-being, and can be critical for energy conservation, metabolic regulation, detoxification, heart function, cognitive function, and memory formation, among other important health considerations. For example, prolonged periods of interrupted or lack of sleep can cause anxiety, stress, depression, and ultimately result in chronic disease. Poor sleep habits in the short-term can likewise cause certain negative effects including, for example, difficulty regulating emotions, reduced alertness and concentration, slower reaction times, decreased vigilance, and decreased impulse control. Sleep consists of two separate, distinct states (i.e., rapid eye movement (REM) sleep and non-rapid eye movement (non-REM) sleep). Both REM and non-REM sleep are defined by unique physiological parameters and are regulated by different parts of the brain. Typically, adults alternate between REM and non-REM sleep throughout the night. These changes between REM and non-REM sleep define a sleep cycle.

Sleep requirements can vary among ages. Although adults typically only require between seven and nine hours of sleep a night, children and adolescents may require between nine and thirteen hours. In some instances, older adults (age 65+) may require even less sleep. Children that do not get enough sleep may struggle to perform effectively in school or at work, and older adults who do not get enough sleep may have difficulty consolidating memories and concentrating on tasks.

The main processes that affect the sleep cycle include (1) the homeostatic process, which is the drive for sleep that accumulates during periods of wakefulness, and (2) the circadian rhythm of the individual which is a regulatory mechanism that follows the light-dark cycle and regulates the sleep-wake thresholds of the homeostatic process. Specifically, environmental cues (e.g., zeitgebers) such as the light-dark cycle of the day can align and affect the circadian rhythm of the individual and in turn affect quality and duration of sleep. For example, excessive light immediately before sleep can inhibit melatonin production which can interfere with the sleep cycle. In addition, the sleep cycle of an individual can be affected by a variety of external behavioral factors, such as staying up late, using electronics before bed, or other environmental concerns.

Poor sleep habits or sleep interruptions can be due to a variety of different causes. These causes can be, for example, biological, environmental, psychological, and behavioral. Individuals may also experience different types of sleep problems, such as difficulty initially falling or staying asleep, insufficient sleep quality, or subsequent difficult waking up. Some individuals also may experience sleep related issues due to lifestyle choices, daily stressors, traumatic events, scheduling challenges, environmental factors and unforeseen events. Environmental factors may include air quality, light intake, noises, and temperature of the habitable space where a person may be trying to sleep, stay asleep or wake up. In addition, behavioral activity affecting sleep quality may include evening caffeine consumption, diet, physical activity, timing of sleep, and the use of electronic devices before bed.

As a result, poor sleep quality can make it more difficult to wake up even after a full night of sleep. When a person's circadian rhythm is in sync with the external world (e.g., the day/night light cycle), the person's body will begin to prepare for waking up which includes a gradual raising of core body temperature, more time spent in lighter sleep stages, and the release of cortisol (a stress hormone to increase energy levels). However, by adhering to a schedule that drastically differs from the day/night cycle, the person's circadian rhythm may be misaligned or otherwise interrupted, and may cause the person to wake up earlier, or later than necessary. Chronic difficulties in waking up can lead to increased fatigue, irritability, and decreased quality of life.

Most people spend significant amounts of time in habitable environments such as enclosed spaces associated with homes, apartments, condominium units, hotel suites or rooms, motel suites or rooms, spas, offices and other work spaces, schools, hospital, and other public and private facilities. Sometimes these enclosed spaces are controlled, or even owned by, the principal occupants, such as homes, apartments or condominium units. Other times these enclosed spaces are controlled by others, for example a facility owner or operator who may own and/or operate a hotel, motel, spa, apartment complex, school, or hospital.

Significant time in these spaces exposes the occupant to a wide range of environmental factors, any of which may have either adverse or beneficial effects on the occupant's health, well-being, or sense of well-being. Monitoring sleep quality, duration, and patterns, as well as being able to identify poor sleep behaviors may help improve a person's health over the long-term. Minimizing exposure to environmental factors that tend to have an adverse effect is desirable, as is increasing exposure to environmental factors that tend to have a beneficial effect. Therefore, it would be desirable to develop new approaches to enhance habitable environments to enhance sleep quality.

### BRIEF DESCRIPTION OF THE DRAWING

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an example habitable space in the form of a bedroom showing sensors for detecting various indicia and controllable devices to be adjusted via a control system for implementing a scene in the habitable space;

FIG. 2 is a schematic diagram of an example control system having a processor, communication circuitry, a memory, and a user interface, the control system configured to adjust operation of devices in a habitable space, and receive detected indicia from sensors associated with the habitable space;

FIG. 3 is a schematic diagram of an example algorithm to be executed by a processor of the control system for adjusting operation of the devices in the habitable space;

FIG. 4 is a flowchart showing the steps of an example “go-to-sleep” scene for encouraging a user to fall asleep at a predetermined time;

FIG. 5 is a graph showing both a linear reduction in light intensity over time and a sigmoid reduction in light intensity over time, the reduction in light intensity occurring before the predetermined bedtime of the user;

FIG. 6 is a graph showing both a linear reduction in light intensity over time and a sigmoid reduction of correlated color temperature over time, the reduction in correlated color temperature occurring before the predetermined bedtime of the user;

FIG. 7 is a flowchart showing the steps of an example “stay asleep” scene for encouraging the user to stay asleep throughout a sleep cycle;

FIG. 8 is a flowchart showing the steps of an example “wake-up” scene for encouraging the user to wake up at a predetermined time;

FIG. 9 is a graph showing both a linear increase in light intensity over time and a sigmoid increase in light intensity over time, the increase in light intensity occurring before the predetermined wake time of the user;

FIG. 10 is a graph showing both a linear increase in light intensity over time and a sigmoid increase of correlated color temperature over time, the increase in correlated color temperature occurring before the predetermined wake time of the user;

FIG. 11 is a flowchart showing example steps for providing educational tips to the user via the control system;

Elements in the figure are illustrated for simplicity and clarity and have not necessarily been drawn to scale. For example, the dimensions and/or relative positioning of some of the elements in the figure may be exaggerated relative to other elements to help to improve understanding of various embodiments.

### DETAILED DESCRIPTION

Generally speaking, in accordance with the present disclosure, systems and methods for enhancing wellness in a habitable space are provided. In some forms, an example system for enhancing wellness in a habitable space includes a control system having a processor, communication circuitry, and a memory. The communication circuitry of the control system is configured to communicate with one or more devices positioned within and/or related to the habitable space. For example, one or more sensors may also be positioned proximate the habitable space to detect parameters or indicia associated with the habitable space (e.g., biometric characteristics of a user, behavioral characteristics of a user, and environmental characteristics) and communicate the detected indicia to the control system. Based at least in part on the detected indicia, the control system is configured to trigger initiation of a “scene” in the habitable space by communicating a signal to at least one of the devices in the habitable space to adjust operation thereof. In other words, the control system is configured to adjust operation of the devices associated with the habitable space to achieve desired environmental characteristics or scenes within the habitable space. For example, the control system may be configured to communicate signals to the devices associated with the habitable space to adjust a luminaire to control light intensity, adjust window coverings to control natural light exposure, adjust an HVAC system to control temperature, and adjust a speaker to control sound, among others. In other forms, the detected indicia may be configured to trigger the settings for specific parameters associated with a scene or transition to or from a scene, or to end a scene upon detection thereof.

As described herein, a “scene” should be understood to refer to a combination of parameters or variables implemented by a control system (which may include one or more hardware and/or software components)and, in some embodiments, one or more other devices, components, systems or subsystems for adjusting the operation of devices in or associated with the habitable space. For example, a scene could include a certain combination of air temperature, lighting effects, and ambient noise, among other features. By one approach, a scene may include programmed environment parameter settings governing a space or sub-space that may be used at particular times of the day or for particular activities. Illustrative examples of scenes are described in greater detail hereinafter, and further described in PCT/US2017/048,382, filed on Aug. 24, 2017, which is hereby incorporated by reference.

In some forms, a control system may be configured to implement one or more scenes simultaneously, in an overlapping manner, or sequentially to improve sleep hygiene of an individual associated with the habitable space. For example, a “go-to-sleep” scene may include a combination of parameters implemented by the control system in the habitable space to increase the likelihood that a person associated with the habitable space will fall asleep at a certain time, such as gradually reducing light in the habitable space, reducing temperature, reducing speaker volume, among other parameters. Additionally, or alternatively, the control system may be configured to implement a “wake-up scene” to increase the likelihood that a person associated with the habitable space will wake up at a certain time. By one approach, a wake-up scene may include a combination of gradually increasing natural or artificial light in the habitable space, increasing temperature, and other parameters as discussed in further detail hereinafter. These scenes may be used individually or in combination (along with other scenes, such as, a “stay asleep” scene) to improve or enhance an individual's sleep quality. A scene may establish one or more environmental conditions within a space or sub-space (or multiple spaces or sub-spaces) to help a user regulate the user's circadian rhythm and to improve thermal comfort and air quality within the space or sub-space.

In some embodiments, a scene may be operating constantly, during a majority of time, at designated times or intervals (e.g., a “go-to-sleep” scene may initiate shortly before a person's bedtime), for a specific length of time, upon selection by a user or system, upon the occurrence of an event or trigger, etc.

Scenes may be triggered or initiated in the habitable environment or a subspace therein automatically based on a trigger condition being met or a trigger threshold being exceeded. In one illustrative example, the control system may determine that a person associated with the habitable space is ready to go to sleep and implement a go-to-sleep scene therein based on indicia detected by one or more sensors or transducers positioned in the habitable space and communicatively coupled to the control system. Such indicia may include biometric characteristics of the person, behavioral characteristics of the person, and environmental characteristics of the habitable space, and the processor of the system may compare the detected indicia to a respective trigger condition. For example, if one or more light sensors detect that all of the lights in a habitable space have been turned off and the time is between, for example, 8:00 pm and 11:00 pm, the control system may determine that the user is ready for bed and may implement the sleep scene without manual input by the user. Alternatively, the control system may bypass the go-to-sleep scene entirely and begin implementation of a stay asleep scene.

Upon the determination that a specific event or other trigger condition has been met or satisfied, the control system may be configured to trigger or transition to a specific scene in the habitable space and control various characteristics of the environment by adjusting devices therein to implement the scene. Additionally or alternatively, one or more scenes may be triggered or initiated in the habitable space manually based at least in part on a user or other input received at a user interface associated with the control system.

In some embodiments, the control system may be configured to determine the trigger or trigger condition based on detected or received information. For example, the trigger may be determined based on detected biometric data, detected environmental characteristics of the habitable space, behavioral data, a user input received at a user interface, and/or data from an external source. In some forms, the control system may be configured to predict effective triggers based on historical data (e.g., the user performing certain actions shortly before bedtime every night). The control system may be configured to automatically determine a trigger based on a predicted or detected sleep time of a user via a machine learning algorithm. For example, the processor of the control system may be configured to determine a person's ordinary bedtime based on data from the sensors indicating that a bedroom light is turned off at a certain time every night, that the person undergoes a routine before sleeping every night, a reduction in heart rate occurring at the same time every night, among others. So configured, the processor of the control system may determine a trigger for implementing a scene based on the sleep schedule of the person determined via historical data stored in a memory of the control system.

Additionally or alternatively, the control system may be programmed to automatically adjust scenes or trigger conditions over time based on data detected via the sensors positioned in the habitable space through machine learning. For example, if a light sensor detects that a person associated with the habitable environment begins turning off the lights at a new, different time every night, the control system may learn the new time via the machine learning algorithm and adjust the scene to automatically implement a sleep scene at the new, updated time for enhancing sleep quality based upon the learned bedtime of the person. So configured, various aspects of scenes may be altered or adjusted based on historical data detected by the sensors of the system and analyzed via the processor using a machine learning algorithm to improve the sleep quality of the user.

In some forms, a scene may be triggered or set based on preprogrammed user preferences or needs. For example, a user may program or schedule a custom or other desired scene via the user interface of the control system to initiate or play at a particular time that the user typically arrives home from work or school, which be may approximately the same time every day or be different times on different days. Alternatively, the scene may be triggered by a motion detector, location detector, or other sensor or device that determines that the user has arrived at home. In another example, a user can customize some of the scenes described below to better fit their personal preferences, goals or needs. In other forms, a scene may be triggered or set based on usage occasions or other external factors. For example, if a usage occasion arises in which a large number of people are detected entering a space or subspace, a scene can be implemented in which the temperature and humidity are decreased to compensate for the crowd. If certain external factors such as rapidly increasing external temperature and humidity are detected, a scene can be implemented to decrease the internal temperature, close blinds, reduce or change window transparency, decrease internal humidity, etc. in order to preemptively adapt the habitable space to include more desirable characteristics and in an effort to avoid an increase in internal temperature and humidity.

In some embodiments, the control system may be configured to determine, implement, and/or set a transition plan in the habitable space from one scene to another scene. A transition plan may include adjusting operation of one or more controllable devices simultaneously, sequentially, in an over-lapping manner, etc. to help facilitate the scene transition. Similarly a transition plan may change different environmental conditions within a space or sub-space at identical, similar or different transition rates based on need, user preferences, user history, scene use history, schedule, controllable device capabilities, configurations or locations, overall power needs or power availability for the space or sub-space, over power needs or power availability for one or more controlled devices that might implement the scenes or be involved in the transition from one scene to another scene, configuration, condition or design of a space or sub-space, needs or goals of a user, usage occasion, etc. For example, if a user has a limited period of time, if there are influencing external factors (e.g., excessive or unusual heat, humidity, sunlight, or air pollution), etc., the system may shorten or change the transition time or plan from one scene to another scene for one or more of the devices associated with the habitable space. In some instances where shortening or changing the transition may not be possible or useful, the control system may alternatively recommend that a user move to a different room or subspace within the habitable space. By way of example, if a user desires to take a nap and changing the current scene is not possible or useful, the control system may recommend another location for the nap depending on the habitable environment.

In some embodiments, the control system may be configured to initiate a transition between scenes in one or more spaces based on a default or preprogrammed transition (e.g., the go-to-sleep scene is configured or scheduled to automatically transition to the stay asleep scene upon the sleep scene ending or upon detection or other determination that a user has fallen asleep in a space). Additionally or alternatively, a user may select, influence or indicate a desired transition time or plan from one scene to another scene for a space.

In some embodiments, configuration or design of the habitable environment may contribute to how a scene is designed, implemented, changed, and set, among other factors. For example, a space's lighting design (e.g., number, installation, location, of photometrics of light bulbs and fixtures), fenestration design (e.g., number, size, location and layout of windows), interior design (e.g., interior wall color, ceiling color, floor color, furniture color, furniture layout, building materials), floor design, layout, the orientation of the space, shade or blind design (e.g., optional positions, thickness, sound and temperature insulation, and transparency), door design (positions, thickness, sound and temperature insulation, and transparency), among other elements, may impact how a scene is established in the space. These factors also may impact the effectiveness of the scene, how a transition to the scene or away from the scene in the space or sub-space is planned or conducted, how a user may react or benefit from a scene implemented in the space or sub-space, and how a default setting for the scene may be set or implemented for the space or sub-space, among others.

In some embodiments, a scene may be triggered or set for only one sub-space within a bigger space such as a bedroom, home office or kitchen within a home, a single apartment within an apartment complex, or a single classroom within a school. For example, two separate individuals located in a habitable space having multiple subspaces may have different preferences such that it may be desirable to implement or trigger different scenes in each subspace where each person is located (e.g., a relaxation scene may be operating in a bedroom within a house or apartment while an energizing scene may be operating in an office within the house or apartment). Alternatively, a scene may be triggered across multiple sub-spaces within a space, such as a home office and a dining room within an apartment, a bedroom and a living room within a single-family home, or a bathroom and a bedroom within a hotel suite. In one example, a “go-to-sleep” scene may be initiated in both a home office and a bedroom where the person in the habitable space typically spends time in the home office before sleep. The parameters of the go-to-sleep scene in each subspace may alternatively be different such that each subspace may include different lighting intensities or color temperatures, different temperatures, different transitions or transition rates for air temperature or lighting changes, different background noises, etc. So configured, the scene may encourage the person to go to sleep before even entering the bedroom.

In some forms, the control system is configured to adjust a scene to accommodate for changes experienced by the user due to travel to a new location. Thus, the control system may adjust for changes in the diurnal cycle and/or circadian cycle. Other information that may be employed by the control system may include, for example, an age or approximate age of the occupant, which may affect or be related to circadian cycle and the ability to adjust for travel (e.g., “jet lag”).

As described above, one or more internal or external sensors or transducer devices may be provided to determine various parameters, factors, or indicia within the space such as the availability, operating state and functionality of any devices in or near the space, the current scene operating within a space, biometric characteristics of a person in the space, and/or behavioral characteristics of a person in the space, among others. Such sensors and the operation thereof will be discussed in further detail below. Based on the indicia detected by the sensors, the control system may be configured to adjust operation of devices in the habitable space, determine a trigger, initiate a scene, adjust a scene, update scene parameters stored in the memory, etc.

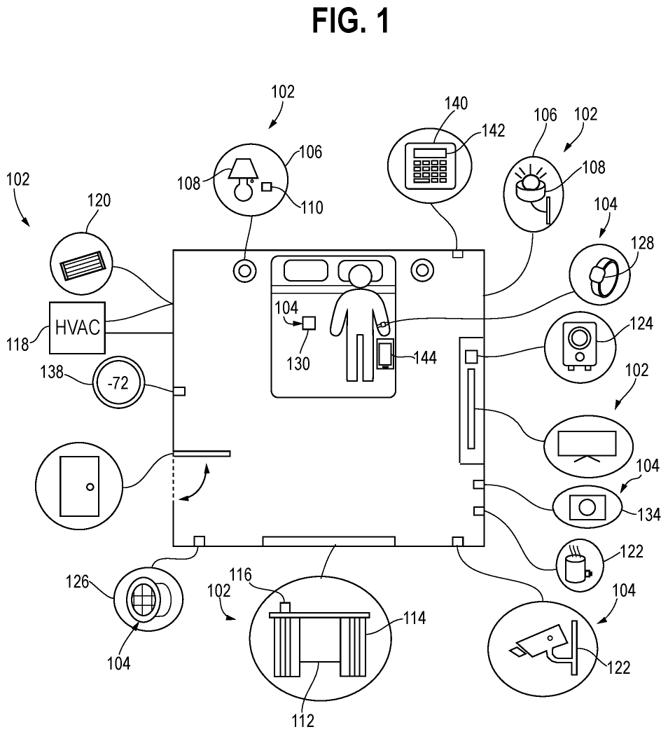

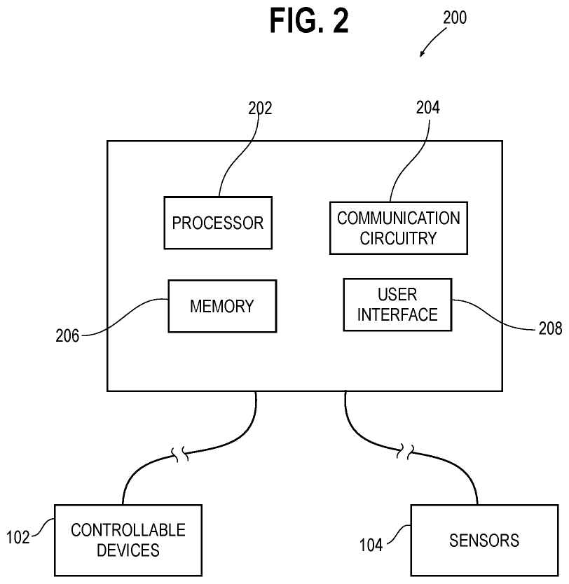

Referring now to the figures, and more specifically FIG. 1, a habitable space 100 is provided including various controllable devices 102 for adjusting various parameters of the habitable space and sensors 104 for detecting various indicia associated with the habitable space 100. The controllable devices 102 may be communicatively coupled to a control system (shown in FIG. 2) such that operation of the devices 102 may be adjusted thereby.

As described above, a number of different controllable devices 102 may be positioned in or near the habitable space 100 to control or adjust one or more various indicia or environmental conditions in or near the space 100. For example, a lighting or luminaire device 106 may be positioned in the habitable space 100 to control the intensity and color temperature of lighting within the habitable space 100. In some forms, the habitable space 100 may include a number of artificial luminaire devices which are controlled by the control system (shown in FIG. 2) to produce a desired output by, for example, varying intensity and/or composition of wavelengths or color of the light. The lighting devices 106 may take a variety of forms. In some forms, the lighting devices 106 may include lamps, sconces, overhead lighting, among others. Additionally, each of the lighting devices 106 may employ a variety of illumination sources 108 such as incandescent lights, florescent lights, compact florescent lights, and light emitting diode (LED) lights. The lighting devices 106 may optionally include ballasts (e.g., electronic ballasts) and/or other electrical or electronic components required for operation. The lighting devices 106 may also include various passive and/or active thermal management components to remove heat, thereby prolonging the operational life of the devices 106

Each lighting device 106 may include a plurality of individual illumination or light sources 108. Additionally, one or more of the lighting devices 106 may be operable to emit light in a respective range of wavelengths at a differing correlated color temperature (CCT). So configured, the lighting devices 106 may be selectively controlled to produce a wide variety of artificial illumination conditions, for instance conditions or scenes that mimic natural light, diurnal light patterns, circadian light patterns, and/or light patterns to accommodate for changes in location (e.g., latitude and/or longitude) or changes in season (e.g., spring, summer, autumn, winter). A circadian light pattern may be a pattern of light during a defined period of time (e.g., solar day, approximately 24 hours) which mimics the intensity and/or color of naturally occurring light (e.g., sunlight and darkness) for a given location (e.g., latitude and/or longitude) and/or at a given time of year (e.g., season, month). A circadian light pattern may be produced by a combination of artificial and naturally occurring light, which may be controlled by the control system to produce said circadian light pattern. The defined or desired circadian light pattern may itself be different from a naturally occurring circadian light pattern at a particular location and/or time of year, or may simply be shifted relative to the naturally occurring circadian light pattern at a particular location and/or time of year. Controlling ingress of ambient light (e.g., sunlight, light from street lamps, buildings or signage, security lighting) from an exterior environment aids in management of exposure to levels of light in order to help maintain healthy circadian rhythms. Accordingly, shades or window coverings 114 discussed below may be employed with these teachings.

In some forms, it may be desirable to reduce the intensity of the light emitted by the lighting devices 106 (or light from external sources outside of the habitable space) before a bedtime of the user associated with the habitable space 100. Each of the lighting devices 106 may include a dimmer or adjuster 110 to gradually reduce the intensity of the lighting shortly before the user's bedtime and may additionally or alternatively alter the CCT of the lighting, as described with respect to the sleep scene discussed hereinafter. The habitable space 100 may further include nightlights, employing dim (e.g., low-wattage) long wavelength LED or incandescent luminaires that engage in response to motion or ambient light levels, and are designed to sufficiently illuminate rooms for safe navigation without disturbing melatonin levels if the user wakes up in the middle of the night.

The habitable space 100 may additionally include controllable devices or components (e.g., window coverings 114) which are controlled to adjust external light being received in the habitable space 100 through one or more windows 112. In one form, the window coverings 114 of the habitable space 100 may not necessarily “cover” the window like shades, and may alternatively be in the form of electrochromic panes placed in the one or more windows 112 to be adjusted by the control system to control a transmissivity of the panes. Additionally or alternatively, the habitable space 100 may include one or more drapes, curtains, shades, or other window coverings 114 coupled to an actuator device 116 such as an electric motor to drive the respective covering 114 along a track to at least partially cover the window 112 in response to the actuator device 116 receiving a signal from the control system. So configured, the control system may regulate the amount of external light received in the habitable space 100. The window coverings 114, such as electrochromic panes in the windows 112, also may be useful in limiting or preventing light from other external sources from penetrating the space, such as artificial light associated with billboards, streetlamps, and/or emergency vehicles, among others. Accordingly, the lighting of the habitable space 100 may be specifically tailored to the sleep goals of the occupants thereof.

The habitable space may additionally include a heating, ventilation, and air-conditioning (HVAC) system 118, some or all of which can be adjusted by the control system for controlling temperature and air quality of the habitable space 100. The habitable space 100 may further include a number of vents 120 that provide air to the habitable space or portions thereof having desired air temperature, humidity, and/or air quality. By adjusting operation of the HVAC system 118, desired environmental parameters or scenes in the habitable space 100 may be achieved. For example, the user associated with the habitable space 100 may prefer a cooler temperature immediately upon arriving home from work. Additionally, there may be health and wellness-based reasons to adjust the temperature, humidity, and air quality of the space (e.g., it may be desirable to lower the temperature in the space to promote decreased core body temperature of the user to prepare the user for sleep). In such forms, the control system may be configured to implement or trigger a scene wherein a thermostat controlling the HVAC system 118 is adjusted by the control system to lower the temperature of the environment in the habitable space 100.

In some embodiments the HVAC system 118 may include regional or multiple zone controls such that the air temperature, humidity, and/or air quality may vary in different rooms or regions of a house or other space or be remediated or changed differently in different rooms or regions of a house or other space. For example, each person in the habitable space 100 may input their preferences generally (e.g., in a user interface of the control system as described with respect to FIG. 2) and for their specific bedrooms. Based on these individual preferences, their associated bedroom 24-hour schedule is set and an average calculation is completed to determine how the temperature should change over time. In addition, each person may also program or otherwise adjust different scenes via the control system to include different a different desired temperature, humidity or air quality caused by the HVAC system 118.

In some forms, a scent dispersing device 122 (e.g., a plug-in scent dispersal device or aromatherapy diffuser) may be provided and controlled to selectively disperse one or more scents into the air in the habitable space 100 or a portion thereof. The scent device 122 may include one or more reservoirs which hold various scents (e.g., lavender and/or rosemary), typically in a liquid or gel form. The scent device 122 may optionally include one or more fans and/or blowers to assist in dispersing the scent(s) into the habitable space 100. The scent device 122 may optionally include one or more heaters (e.g., conductively, radiantly, convectively) thermally coupled to the reservoirs or an output of the reservoirs to heat and thereby vaporize liquid forms of the scent(s) into a gaseous form more easily dispersible into the habitable space 100. The scent dispersing device 122 may be configured to disperse one or more scents in the habitable space 100 upon receiving a signal from the control system (e.g., if the control system implements or triggers a scene including the dispersal of a scent). The habitable space 100 may additionally include an air purifier for purifying air therein.

A speaker or other sound emitting device 124 (e.g., a wireless speaker) may also be associated with the habitable space 100 and be configured or controllable to provide sound into the habitable space 100 or a portion thereof. Similarly, operation of the speaker device 124 may be adjusted upon receiving a signal from the control system. In particular, the speaker device 124 may, for example, provide soothing sounds to mask other sounds (e.g., running water, forest sounds, waves, “white” noise, “pink” noise, music, etc.) while a person in the habitable space 100 is relaxing or is otherwise preparing for sleep. In addition, the speaker device 124 also may provide such soothing sounds during occupant sleep, in one or more transition stages, or upon an occupant waking. In some forms, the speaker device 124 may include one or more speakers which may be positioned throughout the habitable space. Various scenes implemented by the control system may include turning the speaker device 124 on or off, adjusting the content played by the speaker device and/or adjusting the volume of the speaker device. The speaker device 124 may optionally include a nontransitory computer- or processor-readable storage media that stores digital versions of the sounds, for example in a library. The speaker device 124 may additionally include one or more microphones (not shown) to detect noise and potential occupancy in the habitable space 100 and communicate a signal indicative of the detected noise and potential occupancy to the control system. Alternatively, a microphone 126 may be located elsewhere in the habitable space 100. In addition to having a speaker 124, one of the controllable devices 102 also may include other “white” noise generating devices configured to generate soothing sounds.

The control system may be further configured to control operation of a digital screen (e.g., a television 125, a digital alarm clock, a wall mounted smart tablet, etc.) in the habitable space 100 to adjust light output, visual display and sound output by the screen. Additionally, the controllable devices 102 may include a fan for cooling or circulating air within the habitable space 100. Of course, the controllable device 102 as illustrated in FIG. 1 are only examples, and any wired or wirelessly connected device positioned in the habitable space 100 may be adjusted via the control system.

As described above, a number of different sensors or transducer devices 104 may be positioned in the habitable space 100 to monitor various environmental conditions or other indicia in or near the space 100. Such sensors 104 may be communicatively coupled to the control system such that the indicia measured by the sensors 104 may be communicated thereto. In some forms, the sensors 104 are configured to measure various indicia affecting the sleeping environment or sleep hygiene of the user. For example, such indicia could include biometric characteristics (e.g., heart rate, sleep state, respiration rate) of the user obtained from a device worn by the user, behavioral characteristics of the user, environmental characteristics of the habitable space 100, and other factors or characteristics that would otherwise have an impact on the sleep environment or sleep quality of the user. Once identified, such indicia may be measured using the sensors 104 positioned in the habitable space 100, sensors 104 associated with an occupant, or through self-reporting of the occupant or user associated with the habitable space 100. The detected or measured indicia may then be communicated to the control system such that operation of the devices 102 in the environment may be adjusted based at least in part thereon. Additionally or alternatively, the control system may adjust parameters of one or more scenes in the habitable space 100 based at least in part on the data obtained via the sensors 104, as described further below with respect to the control system. So configured, the control system is configured to adjust operation of the devices 102 to improve the sleeping environment or the sleep quality of the user or occupant.

The one or more sensors 104 associated with the habitable space 100 may include various known sensor devices for detecting biometric characteristics of the user to determine a quality of sleep. Such sensors are designed to monitor sleep quality, duration, and patterns to identify poor sleep habits. Relevant biometrics may include any health or wellness-related measurements, including but not limited to heart rate, heart-rate variability, sleep phase, sleep length, respiration rate, walking steps per day, body weight, or BMI. For example, such sensor devices may measure sleep-related indicia via actigraphy. Typical actigraphy devices are based on accelerometers embedded in wearable devices commonly worn on the wrist, ankle, and torso of the user (e.g., wearable device 128). Such measurements may be useful because individuals move less when asleep, and any movement of the person may be subsequently analyzed to extrapolate levels of activity/inactivity, circadian rhythm patterns, and other sleep related characteristics. For example, such sensors could determine poor sleep quality based on excessive tossing and turning throughout the night. Examples of some actigraphy based sleep sensors include FitBit® devices, Garmin® Vivo-Smart, Polar® devices, Samsung® Gear S and the Nokia® Steel HR. Mobile communication devices, such as cell phones, may also incorporate actigraphy based sensors for measuring and extrapolating sleep related indicia of the user.

The one or more sensors for detecting biometric characteristics of the person may also include ballistocardiography (BCG) sensors. Such sensors are configured to measure the movement of the person's body based on the mechanical vibrations caused by cardiac activity to detect and record the user's heart rate and respiration. BCG sensors are unobtrusive, and may include electromechanical film or piezoelectric sensors that translate mechanical energy into electrical signals in a mattress, seat cushion, etc. In the context of the present disclosure, it would be desirable to include a BCG sensor in a mattress (e.g., mattress sensor 130) upon which the user in the habitable space 100 ordinarily sleeps to analyze movement during sleep and assess sleep quality. The measured data may then be extrapolated or otherwise used to derive metrics such as heart rate variability for REM sleep and slow-wave sleep detection. Examples of some BCG sensors include EarlySense® Live, Beddit® 3.0, Nokia® Sleep, EMFIT® QS, RestOn by Terrailon®, and the JA1657 sleep monitor by Joint Chinese Ltd.

Various contactless sleep sensors are also known and may also be used to measure sleep quality of the person by measuring various biometric characteristics of the person. For example, such sensors may be affixed to a wall, nightstand, or other location in the habitable space 100 and measure body movement and breathing patterns throughout the sleep cycle. Such contactless sensors may include cameras incorporating image recognition technology (e.g., camera 132), may corporate sonar technology to determine movement and position of objects or persons in the room, among others. Examples of some contactless sensors include S+ Sleep sensors by ResMed® and SleepScore Max by SleepScore Labs®.

Additionally, the one or more sensors 104 may include various environmental sensors for monitoring characteristics of the habitable space. Based on the indicia or environmental parameters measured by the environmental sensors, the control system may be configured to adjust devices 102 associated with the habitable space 100, or adjust the parameters of a scene in the space 100, as discussed in further detail below. Sensors or transducer devices configured to measure environmental characteristics may include one or more sensors for measuring light intensity (e.g., light detector 134), light color temperature, light distribution, light source location or direction, illuminance, air temperature, air temperature distribution, air pressure, air quality, air movement, air flow source location or direction, air purity (e.g., a particulate detector), water quality, humidity, sound level (e.g., microphone 126), sound distribution, sound source, sound quality, smell or aroma, aroma distribution, occupant number, occupant presence, occupant movement, occupant physical state, occupant clothing color, occupant position within space or sub-space, occupant attribute, orientation, design, shape, presence, color or position of furniture, decorations, ceilings, walls, floors and other features, equipment or materials in a space or sub-space, one or more specific environmental conditions or attributes, amount, presence or absence of one or more chemicals, gases (e.g., carbon monoxide, carbon dioxide), pollutants, pathogens, smoke, micro-organisms, volatile organic compounds (VOCs) or other specific or non-expected materials, operation and location of one or more controllable devices, power use of one or more controllable devices, current scene operating or established in space or sub-space, transition progress from one scene to another scene in a space or sub-space, variation from a scene operating in a space or sub-space, etc. The sensors 104 illustrated in FIG. 1 are only for example purposes, and any sensor or combination of sensors to measure the above non-exhaustive list of indicia may be included in such a habitable space 100.

For example, one or more occupant sensors or detectors (e.g., camera 132) may be positioned in, or proximate the habitable space 100 or portions thereof. The occupant sensor may be configured to detect a presence, or conversely an absence, of an occupant in the habitable space 100. The occupant sensors or detectors may take a variety of forms. For example, the occupant sensor(s) or detector(s) may take the form of various motion detectors, for instance passive infrared based motion detectors, proximity (RF) based motion detectors, microwave or radar based motion detectors, ultrasonic based motion detectors, vibration based motion detectors, and/or video based motion detectors. The occupant sensor(s) or detector(s) may include simple contact switches which detect movement or operation of a fixture or some other element (e.g., turning on a radio, television 125, speaker 124, appliance) by the user. The occupant sensor(s) or detector(s) may take the form of simple cameras (e.g., camera 132) which may capture images, from which changes from frame to frame may indicate a presence or absence of the user The occupant sensor(s) or detector(s) may detect a presence or absence of an object associated with the user, for instance a smartcard or keycard, or a handheld or mobile device. In some forms, the control system may be configured to inhibit the initiation of a scene if one of the occupant sensors indicates an absence of the person in the habitable space 100 such that the scene is not implemented when the user is not home. So configured, power and electricity may be conserved.

In other forms, the one or more environmental sensors may further include one or more temperature sensors or detectors 138 positioned in, or proximate the habitable space 100. Such sensors 138 may be communicatively coupled with the thermostat associated with the HVAC system 118. The temperature sensor 138 may sense or detect a temperature proximate the temperature sensor 138 and provide signals to the control system indicative of the sensed or detected temperature. The temperature sensor 138 may employ various components, for example thermocouples or thermally responsive resistors (i.e., thermistors).

In other forms, the one or more environmental sensors may further include a humidity sensor or detector positioned in, or proximate the habitable space 100. The humidity sensor may sense or detect humidity or relative humidity proximate the humidity sensor and provides signals to the control system indicative of the sensed or detected humidity in the habitable space 100. The humidity sensor(s) or detector(s) may employ various components.

In other forms, the one or more environmental sensors may further include an air quality sensor or detector positioned in, or proximate the habitable space 100. The air quality sensor may be configured to sense or detect, for example, the carbon dioxide concentration of the ambient air in the habitable space 100 and provide signals to the control system indicative of the sensed air quality.

In other forms, the one or more environmental sensors may further include a noise monitor or microphone 126 positioned in, or proximate the habitable space 100. The noise monitor may be configured to sense or detect ambient noise in the habitable space 100 and provide signals to the control system indicative of the sensed noise (e.g., the noise level in decibels).

In other forms, the one or more environmental sensors may further include a photodetector or light detector 134 positioned in or proximate the habitable space 100 or portions thereof to detect the intensity of the light incident therein. For example, the light detector 134 may measure the intensity of the light in the habitable space 100 emitted by the lighting devices 106 (and potentially the light permitted into the space via one or more windows 112) and provide signals to the control system indicative of the sensed light intensity.

Any combination of known sensors 104 may be utilized within the scope of the present disclosure to measure biometric characteristics of the user, behavioral characteristics of the user, and environmental characteristics of the habitable space 100 depending on the desired characteristics to be measured. Based on the characteristics measured by the sensors 104, the control system may be configured to cause initiation of a scene in the habitable space 100, adjust operation of the devices 102 in the habitable space 100, or adjust a scene to be implemented in the habitable space 100.

The control system for the habitable space, and operation thereof, will be described in more detail with reference to FIG. 2. As illustrated, the control system 200 includes a processor 202, communication circuitry 204, a memory 206, and a user interface 208. As described above, the control system 200 may also be communicatively coupled with one or more of the controllable devices 102 positioned in the habitable space 100 and may further be communicatively coupled with the sensors 104 positioned in the habitable space 100 to receive signals therefrom. The control system 200 may take the form of a programmed computer or other processor-based system or device. For example, the control system 200 may take the form of a conventional mainframe computer, mini-computer, workstation computer, personal computer (desktop or laptop), or handheld computer. Additionally, the control system may be communicatively coupled to a remote server computer such that processing operations of the control system 200 are performed by the remote server computer. Non-limiting examples of commercially available computer systems include, but are not limited to, an 80×86, Pentium, or i7 series microprocessor from Intel Corporation, U.S.A., a PowerPC microprocessor from IBM, a Sparc microprocessor from Sun Microsystems, Inc., a PA-RISC series microprocessor from Hewlett-Packard Company, or a 68xxx series microprocessor from Motorola Corporation.

The control system 200 is configured to operate in a networked environment via the communication circuitry 204 thereof. Specifically, processor 202 may be configured to cause the communication circuitry 204 to communicate signals to the devices 102 in the habitable space 100 over a network such as the internet or through a local connection, and the communication circuitry is additionally configured to receive signals from other devices, such as the one or more sensors 104 described above. In other forms, the communication circuitry 204 may communicate with the devices 102 via a short-range communication protocol such as Bluetooth®. Such communications may be made via wired and/or wireless network architectures, for instance, wired and wireless enterprise-wide computer networks, intranets, extranets, and the Internet. Thus, the communication circuitry 204 may include wireless communications components, for example one or more transceivers or radios and associated antenna(s) for wireless communications (e.g., radio or microwave frequency communications, collected referred to herein as RF communications). Other embodiments may include other types of communication networks including telecommunications networks, cellular networks, paging networks, and other mobile networks.

As described above, the control system 200 may include one or more processors 202 and software components. The processor 202 may be any logic processing unit such as a central processing unit (CPU), digital signal processor (DSP), application-specific integrated circuit (ASIC), field programmable gate array (FPGA), programmable logic controllers (PLC), among others.

The control system 200 may further include a memory 206. The memory 206 may include nontransitory Flash or read-only memory (“ROM”) and nontransitory random access memory (“RAM”). Various program engines can be stored in the memory 206, such as an operating system, one or more executable algorithms, one or more application programs, other programs or engines and program data. Application programs may include instructions that cause the processor 202 to automatically generate signals to control various parameters or scenes in the habitable space 100 based on, for example, one or more sensed indicia of the habitable space 100 itself or the user therein. Additionally, application programs may include instructions that cause the processor 202 to automatically receive input and/or display output via the user interface 208.

The user interface 208 of the control system 200 may encompass a various of input/output (I/O) interfaces such as a wall mounted interface (e.g., interface 140 in FIG. 1) or screen positioned in the habitable space 100, a voice operated home assistant (e.g., Amazon's Alexa®), a keyboard, a graphical user interface (GUI), a physical switch, a network-connected “smart” switch, and/or a touchscreen. In some forms, the user interface 208 may be a touch screen on a mobile communication device (e.g., cellular smart phone) of the user. In other forms, the user interface 208 can include a microphone, a joystick, a game pad, a tablet, a scanner, among others. The user interface 208 is configured to receive a user input from the user associated with the habitable space 100 and may be configured to store the user input in the memory 206 of the control system 200. In some forms, the user input may be configured to cause initiation of a scene in the habitable space 100, cause adjustment of the devices 102 in the habitable space 100, and/or adjust the parameters of a scene to be implemented in the habitable space 100.

For example, a user operable wall-mounted interface 140 in the habitable space 100 may include a display 142 (e.g., LCD) to display various information and characteristics of the habitable space 100. The user operable wall-mounted interface 140 (or any disclosed user input) may further include user actuatable controls (e.g., user selectable icons displayed on touch screen, keys, buttons) manipulation of which allows the user associated with the habitable space 100 to select parameters or scenes to cause the control system 200 to adjust one or more of the environmental parameters or scenes in the habitable space 100. For example, the user may be able to manipulate the actuatable controls to change the temperature in the habitable space 100, adjust the humidity, adjust the intensity of the lighting, among others.

In another form, a mobile or handheld device (e.g., smart phone 144 of the user shown in FIG. 1) may serve as the user interface 208. The mobile or handheld device may include a display (e.g., LCD) to display various information and characteristics of the habitable space 100. The device may further include user actuatable controls (e.g., user selectable icons, keys, buttons) manipulation of which allows the user associated with the habitable space 100 to select parameters or scenes to cause the control system 100 to adjust one or more of the environmental parameters or scenes in the habitable space 100. Additionally, the mobile or handheld device may execute a downloaded customized application or “app” that communicatively interfaces with the control system via a wireless connection protocol (e.g., IEEE 802.11, BLUETOOTH®, WI-FI®). So configured, the user can access the controls and various information regarding the characteristics of the habitable space 100, the scenes in the habitable space, and indicia measured by the sensors 104.

The user interface 208 may also allow or facilitate collection of information from the user (e.g., during an initial setup of the system) which is indicative of the user's behavioral patterns, the user's impressions of their own sleep quality, and the user's overall satisfaction with the habitable space 100. Such may be captured with an administered survey, which includes various questions and possible ratings, presented for instance via a graphical user interface (GUI). In some forms, the control system 200 may administer an initial survey before initiating any configured scenes in the habitable space 100 to assess various preferences and characteristics of a user. For example, before initiating or transitioning to sleep-related scenes it may be desirable to obtain information regarding behavioral characteristics such as the ordinary sleep schedule of the user, background information on sleep habits, or baseline sleep characteristics (e.g., the quantity of sleep the user gets on weekdays and weekends, how sleepy the user is during the daytime, whether the user self-reports any sleep issues or health issues that might otherwise affect sleep, how often the user uses an alarm, a sleep time of the user, a wake time of the user, etc.). It may also be beneficial to detect ambient characteristics of the user's bedroom during sleep time such as temperature, light, and noise (e.g., via the one or more sensors 104 described above).

The survey may include a number of questions concerning the user's perceived quality of sleep. Additionally or alternatively, the survey may include daily multiple-choice questions regarding sleep quality providing feedback opportunities for the user in the habitable space 100. In one example, a question presented to a user may ask “how would you rate your overall sleep quality?” with the answers (a) very good, (b) good, (c) poor, and (d) very poor. In some forms, the questions may be short answer questions.

In still other forms, the user input may include a sleep schedule change of the user. For example, after a sleep schedule for the user has been established (e.g., by detecting various indicia indicating a sleep time of the user or a user input indicating the sleep time of the user) the user may input a change in the sleep schedule to adjust the sleep schedule previously detected or received by the control system.

The control system 200 may be configured to adjust parameters of various scenes based at least in part on the responses by the user. For example, the user input may include one or more sleep-related goals of the user and the control system 200 may adjust the parameters of scenes to accommodate those goals. If the user indicates that he or she prefers to sleep in on weekends, the control system 200 may delay the initiation of a wake-up scene in the habitable space 100 that would otherwise encourage the user to wake up at a specific time. If the user indicates that he or she has a goal of waking up an hour earlier every morning, the control system 200 may be configured to trigger initiation of a wake-up scene in the habitable space 100 at an earlier time. In another example, if the user indicates sleep issues resulting from extensive light exposure before bed, the control system 200 may be configured to dim the lighting devices 106 in the habitable space 100 during a go-to-sleep scene and additionally dim the screen of the user's smart phone 144 (or television 125, among other screens) such that the light in the habitable space 100 does not suppress the user's melatonin production before sleep.

The user interface 208 may also request information regarding various behavioral characteristics of the user such that the control system 200 may adjust various parameters of the habitable space 100 based at least in part thereon. Behavioral factors of the user can influence sleep quality, but such behavioral characteristics may be difficult, or impossible, to measure via the sensors 104 associated with the habitable space 100. Such behavioral factors affecting sleep quality may include alcohol consumption, caffeine consumption, times for eating meals, the amount, type and quality of food eaten, physical activity, and timing of sleep onset (all of which may occur outside of the habitable space). To assess and analyze these behavioral characteristics, the user may respond to requests on the user interface 208 or may proactively input or otherwise provide information to be analyzed by the control system 200. So configured, the control system 200 can adjust parameters of the habitable space 100 or adjust parameters of scenes to implement in the habitable space 100 based at least in part on the behavioral response of the user.

The user interface 208 may further provide capabilities for the user to manually adjust operation of the control system 200, similar to a universal remote. In some embodiments where the user interface 208 is a touch-sensitive or touch-responsive display with a GUI, the GUI may include one or more user selectable icons (e.g., scroll bars, tool bars, pull down menus, dialog boxes, keys, and/or text) displayed for selection by the person. Selection may allow the user to adjust illumination, temperature, humidity, sound, and/or other aspects of the space 100 or otherwise adjust or initiate a scene in the habitable space 100. The GUI may present the user with a set of defined programs or scenes to select from. The GUI may optionally allow the person to define new programs or scenes, delete old programs or scenes, and/or modify existing programs or scenes. In addition, the GUI may provide an illustrated representation of the environment showing the various devices to be adjusted in the habitable space 100 such that a user may select each device to obtain a status thereof (e.g., battery percentage, wireless connectivity, etc.) or adjust operation thereof.

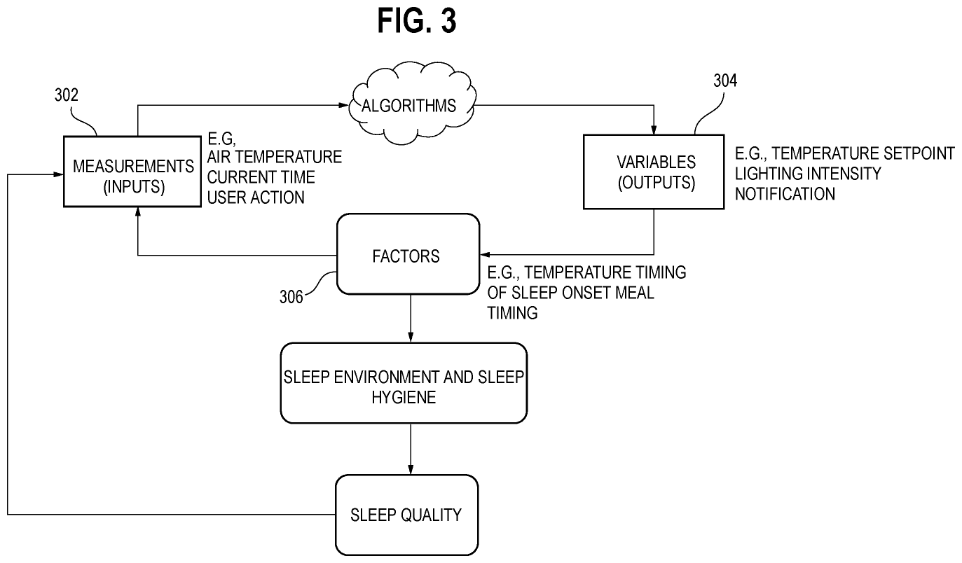

The control system 200 may be configured to generate a new program or scene, or execute an existing program or scene with one or more new or modified parameters, hence in effect constituting a new program or scene. Such adjustments may be based on a machine learning algorithm as described below with respect to FIG. 3. For instance, the control system 200 may determine that the occupant has recently traveled from a location with a significantly different natural light cycle from that of the location of the habitable space 100. Thus, the control system 200 may automatically select a program or scene which provides specific illumination or other characteristics that alleviates or otherwise addresses symptoms or ailments associated with such changes in natural illumination due to the travel, such as jet lag. Scenes may also be adjusted by the control system 200 based at least in part on the indicia detected by the one or more sensors 104, as described in further detail below. If a change has been made by the control system 200, the control system 200 runs the new program or scene with new parameters to provide adjusted environmental characteristics or scenes. Execution of the new program causes the control system 200 to adjust the operation of the devices 102 positioned in the habitable space 100 to provide the environmental characteristics, scenes or amenities in the habitable space 100 in accordance with the new, updated parameters.

In some embodiments, the control system 200 is configured to trigger or initiate a scene in response to a prerequisite trigger condition being satisfied. Such a “trigger” may be indicative of a sleep time or a wake time for a person such that a go-to-sleep scene or a wake-up scene, respectively, may be initiated. These “triggering” events represent a judgment in a logical decision-making process by the processor 202 of the control system 200. In some forms, the control system 200 may be configured to determine a trigger for a scene based on the detected indicia. When the requirements of the trigger have been satisfied, the control system 200 will automatically initiate the scene. Otherwise, the scene will typically not initiate unless the trigger condition is satisfied. In some forms, the control system 200 is configured to trigger initiation of a scene based at least in part on the indicia measured by the one or more sensors 104. For example, the scene could be initiated based on one or more of the detected biometric characteristics of the user, the behavioral characteristics of the user, and the environmental characteristics of the habitable space 100. In other forms, the control system 200 is configured to manually initiate a scene based at least in part on user input received at the user interface 208 of the control system 200 (e.g., a user indicates or schedules a desired sleep or wake time). In still other forms, the trigger may be based on data from an external source, such as weather data, a work schedule of a user from a calendar application, a recommendation from a doctor, etc. The trigger conditions may be the same, or different, for any number of scenes configured to be implemented by the control system 200. Various trigger conditions will be discussed in more detail below with respect to example scenes implemented by the control system.

An example flowchart diagramming the operation of a process by the control system 200 is illustrated in FIG. 3. As shown at 302, a number of measurements (e.g., the indicia detected by the sensors 104) constitute the inputs for an algorithm. For example, the detected indicia may include the temperature detected by the temperature sensor 138, the noise level detected by the microphone 126, and/or detection of the user by the camera 132. The processor 202 of the control system 200 may then compute the variables shown at 304 (e.g., the adjustments in operation of the devices 102 in the habitable space 100) based on the inputs described above.

As shown at 306, any number of factors or indicia may be selected for measurement and adjustment. In some forms, the algorithm implemented by the processor 202 may be configured to measure and adjust temperature in the habitable space 100. In other forms, the user may select any number of factors or indicia to monitor before, during, and after the implementation of a scene. These measured factors or indicia are characteristics that affect the sleep environment in the habitable space 100 and the sleep quality of the user. Based on the sleep quality (e.g., as assessed via user input in the user interface 208), the control system 200 may be further configured to analyze user preference and satisfaction as inputs to the algorithm to account for the user's desired environmental parameters during scenes.

Various example scenes will now be described in accordance with the present disclosure, and can be implemented and adjusted via the control system 200 and the methods described herein. These are only intended as illustrative examples. Any number of scenes having different combinations of parameters may be implemented in the habitable space as desired by the user. Similarly, the scenes below can be altered and still achieve the desired effect.

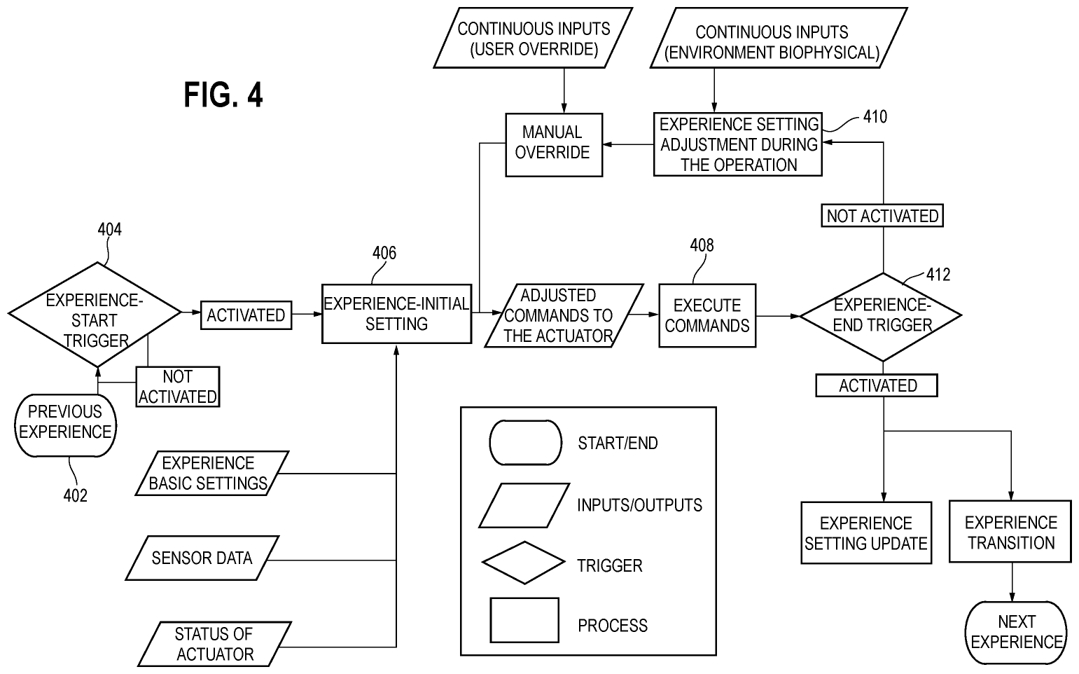

For example, in some embodiments, the control system 200 may be configured to implement a scene to improve the user's sleep environment and hygiene before and during the time of going to sleep. Specifically, FIG. 4 shows a logic flowchart that may be followed by the control system 200 while implementing the scene. Such a scene may be referred to hereinafter as a “go-to-sleep” or “ready for sleep” scene. The go-to-sleep scene is configured to improve sleep hygiene of the user by gradually preparing the user for sleep by, for example, automating sleep cues by adjusting operation of devices 102 associated with the habitable space 100 to adjust the environmental parameters therein. In some forms, the sleep cues for the go-to-sleep scene may include a gradual reduction in light intensity, a gradual reduction in speaker intensity, a gradual reduction in temperature, among other factors conducive to improving sleep quality.

As described, the control system 200 may initiate the go-to-sleep scene at a predetermined time before the user's desired bedtime such that the user may be transitioned from an awake state to a sleeping state. For example, the scene may be triggered or initiated based on a preset clock, a preset time on a calendar for sleep, a scheduled bedtime, among others. In some forms, the control system 200 may initiate the go-to-sleep scene about 15 minutes before a desired bedtime of the user, about 30 minutes before a desired bedtime of the user, or about 45 minutes before a desired bedtime of the user, among others. So configured, during the time period in which the go-to-sleep scene is implemented, there may be a gradual transition to a sleeping state of the user to improve the sleep quality thereof.

In other forms, the control system 200 is configured to initiate the go-to-sleep scene as shown at step 404 based upon a predetermined trigger condition being met or a trigger threshold being exceeded. Such a trigger could include completion of a previous scene, detected biometric characteristics of the user, detected environmental characteristics of the habitable space 100, detected behavioral characteristics of the user, a detected user input, and/or data from an external source. In some embodiments, the trigger may be a combination of conditions, such as, for example, a detected location of a user, completion of a previous scene, and a detected activity level of the user.

In some forms, as shown at 402, the control system 200 may be configured to initiate the go-to-sleep scene upon completion of another scene within the habitable space 100. For example, the user may have selected a different scene shortly before bedtime and the control system 200 may transition between from the different scene to the go-to-sleep scene automatically upon the trigger condition being satisfied. If the trigger condition for the go-to-sleep scene is not satisfied, the control system 200 may maintain the parameters of the previous scene.

The trigger to initiate the scene may be determined by the control system 200, and may be based at least in part upon the biometric characteristics of the user detected by the one or more sensors 104. For example, the go-to-sleep scene may be triggered by a reduction in movement, heart rate, and/or breathing of the user measured by one or more wearable devices (e.g., wearable 128), an estimated or self-reported fatigue level based on physical activity of the user, and other measurements indicating sleep readiness.

Additionally or alternatively, the start trigger may be based at least in part upon the environmental characteristics of the habitable space 100 detected by the one or more sensors 104. For example, the go-to-sleep scene may be triggered by detection of a specific scent (e.g., lavender) being released into the space 100, a voice command, a decrease in room temperature, detection of a person entering the bedroom, detection of a person stopping internet access, detection of a reduction in usage of electricity, detection of a person laying on a mattress or sofa (e.g., by the sensor 130 shown in FIG. 1), detection of a person arriving home, detection of a person taking a shower, detection of the lights being turned off, detection of a lack of noise, detection of any other event indicating that the user has started to routine before going to bed (e.g., brushing teeth, playing relaxing music, turning off music, shutting bedroom door, etc.), a stable pressure is detected on a bed surface, detection of the user making a specific gesture, a signal or communication from a voice assistant, etc. In some embodiments, the go-to-sleep scene may be triggered by an event, but may begin a period of time after detection of one of the above. Accordingly, in one example, the go-to-sleep scene may begin 45-minutes after completion of an evening shower and teeth brushing.

The start trigger may also be based at least in part upon the behavioral characteristics of the user either detected by the one or more sensors 104 or reported via the user interface 208 by the person. For example, the go-to-sleep scene may be triggered by the user reporting via the user interface 208 that he or she is very tired, etc.

Various other triggers may be based at least in part on data from an external source to determine when to initiate the go-to-sleep scene. For example, the go-to-sleep scene may be triggered by instruction from a third party, user input from a family member believing the user is tired (which may be particularly useful for young children), data from a weather application, a work schedule of the user from an application on a computer or phone, among others. In addition, the control system 200 may be configured to adjust the start trigger depending on changes in time zones (e.g., to accommodate for jet lag), and also changes in daylight savings time.

In some forms, combinations of detected triggers may be analyzed to determine whether to initiate the go-to-sleep scene. For example, initiation of the go-to-sleep scene may require multiple trigger conditions being met, such as both a detection that the user's heart rate has declined in addition to a detection of the lighting devices 106 being turned off. So configured, the control system 200 can increase the confidence interval that the user in the habitable space 100 is actually getting ready to go to sleep as opposed to just turning off lights when leaving a room.

In still other forms, the control system 200 may be configured to monitor the user via the sensors 104 over an extended period of time such that the control system 200 may be able to predict a user's bedtime based on historical data stored in a memory thereof and may be configured to trigger the go-to-sleep scene at a time based on the prediction. For example, if the control system 200 detects that the user goes to sleep between 10:00 PM and 11:00 PM every night, the control system 200 may determine that it should trigger the go-to-sleep scene between 9:30 PM-9:45 PM. Such a prediction may be based on, for example, an average bedtime of the user over a prior 30-day period as detected by the sensors 104. Such a predicted bedtime also may be used in addition with the triggers, such that the control system 200 may automatically initiate the go-to-sleep scene after the user or occupant has brushed their teeth within a window of 9:30 PM-10:45 PM, if the user typically goes to bed between 10:00 PM and 11:00 PM.

In addition, various other triggers or trigger conditions may be configured to cause a delay of the onset of the go-to-sleep scene upon detection by the control system 200. Triggers also may be scheduled so that they are active all the time or only some of the time (e.g., during the work week but not on the weekend). The user may not always be able to go to sleep at the same time every night, and the control system 200 may be able to adjust the go-to-sleep scene based at least in part on indicia detected by the one or more sensors 104, and the control system 200 may inform the user of a delay. For example, the go-to-sleep scene may be delayed upon a detection or other determination that the habitable space 100 is not occupied, detection that the user has left the bedroom, detection that a person who does not usually sleep in a specific bedroom is in the bedroom, determination that the user's schedule has changed, determination that the user will be traveling to a different time zone, detection that a person is cooking, detection that the user is talking, a recommendation from a third party to stay up later, detection of an event occurring, detection of a specific activity (e.g., baby crying, shower still running, increase in noise level, increase in talking, increase in light level), detection that the user has not yet completed an activity usually done before trying to go to sleep (e.g., brushing teeth, going to bathroom, taking a shower, taking medication, setting an alarm, texting message to certain person), detection of a manual user input indicating that the scene should be delayed, detection of a window or door opening, detection of an emergency event, and/or detection of the user having trouble falling asleep (e.g., tossing and turning detected by a mattress-based sensor), etc.

The go-to-sleep scene can also be manually disabled by the user through the user interface 208 associated with the control system 200. In some forms, the go-to-sleep scene may be inhibited or delayed from triggering if the user is not detected in the habitable space 100 (e.g., by the occupant-based sensors such as the camera 132 described above). Additionally, or alternatively, the go-to-sleep scene may be inhibited from triggering if the control system 200 detects that the user is already asleep. In such an instance, the control system 200 may be configured to instead initiate a stay asleep scene, as described below.

Once the trigger is satisfied, at step 406, the control system 200 begins to initiate the go-to-sleep scene by communicating signals to the devices 102 associated with the habitable space 100 based on the basic or initial settings (i.e., from setting up the system 200), indicia detected by the one or more sensors 104, and the status of the devices 102. The basic settings for the go-to-sleep scene may either be default settings based on current scientific research regarding beneficial environmental settings, or may be input by the user at the user interface 208 during an initial installation or registration to reflect preferences of the user. For example, the user may input a typical bedtime, a typical wakeup time, and desired operation of the devices 102 in the habitable space 100 as described below.

The basic settings may include a desired light intensity during the go-to-sleep scene such that the lighting devices 106 in the habitable space 100 may be adjusted by the control system 200 to the desired intensity either rapidly or gradually over a period of time. As described herein, light intensity is the percentage of lumens output from a lighting device 106 in the habitable space 100. The desired light intensity before sleep may be set to a default of 10% light intensity and may be adjusted by the user in the range of 0-50% light intensity (or lower or higher percentage depending on the maximum light intensity that is possible for the desired light). For example, in some forms, the user may adjust the light intensity beyond 50%, but the control system 200 may notify the user that a light intensity over 50% falls outside of the predetermined range (e.g., based on current scientific research). Once the go-to-sleep scene is initiated, the lighting devices 106 may start gradually changing from either the current light intensity (e.g., 100%) or 50% light intensity of the lighting devices 106 in the habitable space 100, whichever is lower.

The basic settings may also include a desired correlated color temperature (“CCT”) during the go-to-sleep scene such that the lighting devices 106 in the habitable space 100 may be adjusted by the control system 200 to a desired CCT either rapidly or gradually over a period of time. As described herein, CCT is the specification of the color appearance of the light emitted by a lighting device 106, relating to its color of light from a reference source when heated to a particular temperature, measured in degrees Kelvin (K). In one illustrative approach, the desired CCT before sleep may be set to a default 2000K and may be adjusted by the user in the range of 1400-4000K. Once the go-to-sleep scene is initiated, by some approaches, the lighting devices 106 will start changing from either the current CCT or 2700K in the habitable space 100, whichever is lower.

In embodiments including a window shade or covering 114 that may be controlled via an actuator device 116, upon initiation of the go-to-sleep scene, the control system 200 may cause the actuator device 116 to close the shade or covering 114 so as to reduce the light present in the habitable space 100. In some forms, the settings for the window shade or covering 114 may be binary (i.e., open or closed). In other forms, the settings for the window shade or covering 114 may provide a sliding scale such that the covering 114 may only be partially closed and may still let some natural light (e.g., from the moon) into the habitable space 100. In some embodiments, the electrochromic glass or panes may be employed to slowly decrease the amount of external light being permitted into the habitable space via the window 112.

The basic settings may also include a desired temperature setpoint for the habitable space 100 during the go-to-sleep scene such that the HVAC system 118 or thermostat may be adjusted by the control system 200 to a desired temperature. By way of example, the desired temperature reduction before sleep may be set to a default of 2° C. and may be adjusted by the user in the range of 0-5° C. In other forms, the user may adjust the temperature outside of the 0-5° C., but may be notified by the control system 200 that the user's selection falls outside of the predetermined range. Once the go-to-sleep scene is initiated, the control system 200 will cause the HVAC system 118 to start changing the temperature in the habitable space 100 to the desired temperature, which may be a lower temperature. For example, the control system 200 may cause the temperature to change from about 27° C. to about 25° C.

In embodiments where the habitable space 100 includes one or more speaker devices 124, the basic settings may also include a desired speaker intensity during the go-to-sleep scene. As described herein, speaker intensity is the percentage of decibels output from a speaker device 124 in the habitable space 100. The desired speaker intensity before sleep may be set to a default of 25% and may be adjusted by the user in the range of 0-50%. In other forms, the user may adjust the speaker intensity above 50%, but may be notified by the control system 200 that the user's selection falls outside of the predetermined range. Once the go-to-sleep scene is initiated, the control system 200 will cause the speaker device 124 to start changing the speaker intensity in the habitable space 100 to the desired intensity. During initial setup, the user may also select the desired speaker content. In some forms, the one or more speaker devices 124 may be configured to play soothing sounds, such as white noise, nature sounds, songs, or other music selected by the user that are conducive to improving sleep quality. The speaker device 124 may be configured to interface with a streaming service such that the user may select a song or other music to play thereon. If the content selected by the user is no longer available (e.g., file missing, license expired, etc.), the control system 200 may select a default sound and notify the user that the selected content is not available.

The basic settings may also provide an option for desired notifications and reminders for the user regarding the go-to-sleep scene. Upon initiation of or during the go-to-sleep scene, the control system 200 may be configured to push one or more notifications or reminders to the user. Such notifications may include helpful tips concerning sleep quality, may include reminders (e.g., “15 minutes until bedtime”), among other suggestions to encourage behavior change towards achieving better sleep preparation. The desired frequency (i.e., how often the notifications are pushed) may be set to a default of one every hour and may be adjusted by the user (e.g., every 30 minutes, every 5 minutes, etc.). In addition, the timing of notifications and reminders before sleep may be set to a default of, for example, 15 minutes before bedtime, and may be adjusted by the user (e.g., 5 minutes before bedtime, every 10 minutes over a 30 minute interval before bedtime, etc.). So configured, the user may customize to what extent and when and how they receive notifications. Alternatively, such notifications may be configured to only appear upon interaction of the user with the control system 200 (e.g., through the user interface 208). These notifications can be delivered to the user through multiple approaches, such as being communicated or “pushed” to the smart phone 144 of the user, smart watch, smart band, communicated by a voice assistant, etc.

The basic settings may also include a desired brightness of electronic devices associated with the habitable space 100 during the go-to-sleep scene such that the displays of such electronic devices (e.g., the smart phone 144, a television, etc.) in the habitable space 100 may be adjusted by the control system 200 to a desired brightness. Alternatively, if direct adjustment of the electronic devices is not possible, the control system 200 may push a notification to the user to recommend that the user lower the brightness on any digital devices being used. It is known that excessive brightness from electronic devices, such as smart phones, prior to sleep can suppress melatonin production by the suprachiasmatic nucleus, thus reducing the quality of sleep. The control system 200 may be configured to communicate signals to the electronic devices in the habitable space 100 to adjust brightness thereof during the go-to-sleep scene. The desired brightness before sleep may be set to a default of 50% and may be adjusted by the user in the range of 0-100%. So configured, the control system 200 can cause the brightness of the screen on mobile and wearable devices, and any other electronic screens in the habitable space 100, to be dimmed.

The status of the devices 102 may also be continuously verified by the control system 200 such that if a device is broken or inoperable the control system 200 will not include operation of that device in the scene. For example, if the control system 200 detects that one of the lighting devices 106 has a bulb burnt out, the control system 200 may not communicate a signal to adjust operation of the lighting device 106.

The basic or default settings may be preprogrammed in the memory of the control system 200 based on, for example, current scientific literature or sleep related research. In some forms, communication circuitry 204 of the control system 200 may be configured to receive updates from a remote server computer or other source to adjust such stock settings over time. As noted above, users may thereafter modify these settings via the user interface 208 of the control system 200, such as through an application on their smart phone or other mobile communication device.

The control system 200 may then communicate signals to the controllable devices 102 at step 408 to adjust the devices 102 according to the selected or triggered scene based on the initial settings described above.

After the control system 200 has caused initiation of the go-to-sleep scene and the basic settings for one or more of the devices 102 associated with the habitable space 100 have been implemented via the signals from the communication circuitry 204, at step 410, the control system 200 may be configured to continue measuring various indicia in the habitable space 100 via the one or more sensors 104 to adjust the scene over time. For example, in embodiments where the go-to-sleep scene is initiated about 30 minutes before the bedtime of the user, the control system 200 may continuously monitor the data obtained via the sensors 104 over the about 30 minute interval to gradually adjust operation of the devices 102 in the habitable space 100 to provide a smooth transition between an awake state and an asleep state. In other forms, the control system 200 may monitor and adjust operation of devices 102 over only a portion of the go-to-sleep scene.

In one form, the control system 200 may monitor the light intensity in the habitable space 100 via the light sensor 134 and gradually reduce the light intensity during the go-to-sleep scene by sending one or more signals to the lighting devices 106. In the default form, the light intensity may be reduced from about 100% to about 10% during the go-to-sleep scene and can be reduced at a variety of speeds or in different intervals selected by the user. Depending on the lighting devices 106 associated with the habitable space 100, the control system 200 may adjust the lighting intensity in a linear manner, or in some forms, can adjust the lighting intensity according to other non-linear functions.

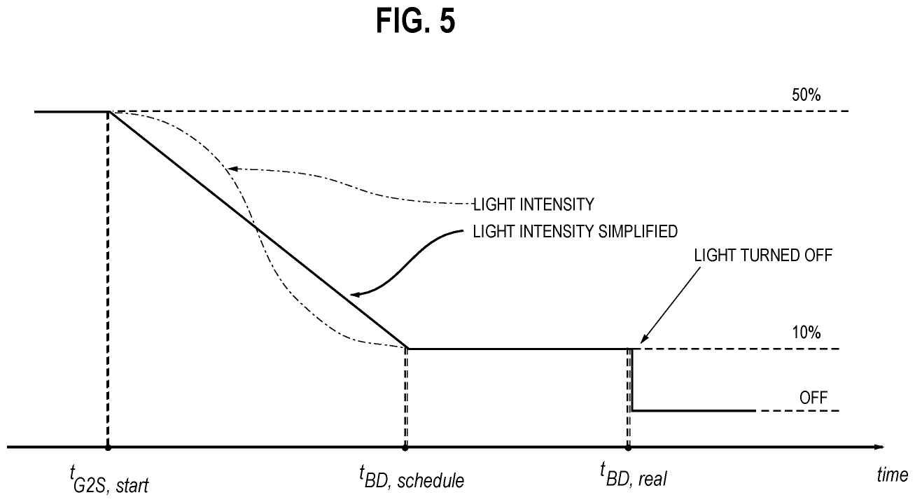

FIG. 5 is a an example graph showing the change in light intensity of the lighting devices 106 by the control system 200 according to both a sigmoid transition and a linear transition. As shown, the light intensity is reduced from its initial intensity (for example, a default may be set to 50% of pre-scene light intensity) to its intensity at the end of the go-to-sleep scene (for example, a default may be set to 10% of pre-scene light intensity). As shown, the adjustment would occur at a specified rate between initiation of the go-to-sleep scene and the scheduled or predicted bedtime of the user. In the illustrated form, the following sigmoid function may be used to gradually reduce the light intensity in the habitable space as shown in accordance with FIG. 5

$$LI ( t ) = \frac{1}{1 + {e}^{k ( \frac{t - {t}_{G 2 S , start}}{{t}_{G 2 S} - {t}_{LI , end}} - \frac{1}{2} )}} ( {LI}_{init} - {LI}_{end} ) + {LI}_{end}$$

In the above function, t is the time during the go-to-sleep scene, t_{G2S,start} is the time the go-to-sleep scene is initiated by the control system, t_{G2S} is the amount of time the go-to-sleep scene should start before the bedtime of the user (e.g., a default of 30 minutes), LI_{end} is the light intensity at the end stage of the go-to-sleep scene, which is also the light intensity at the bed time, and LIᵢₙᵢₜ is the initial light intensity. These values can be set by the user in the basic settings. Generally, the value of LI_{end} is lower than LIᵢₙᵢₜ, LI_{end} will be set as LIᵢₙᵢₜ. Additionally, t_{LI,end} is the duration the final light intensity will last. By one approach, the default value of t_{LI,end} is (t_{G25}−20) min, which typically provides enough time for light intensity transition (20 mins). Further, k is the steepness of the sigmoid function, which also impacts the smoothness at the beginning and ending of the light intensity transition. In some configurations, the default value of k is 7, which can be updated after user feedback or otherwise modified by the user. Such values can also be updated based on the detected indicia in the habitable space 100 by the one or more sensors 104 as described above.

If the sigmoid function identified above cannot be implemented in the lighting devices 106 in the habitable space 100 (e.g., there is no dimmer, or means for gradually reducing the light intensity over a period of time), a simplified linear change of light intensity may be implemented using the equation:

$$LI ( t ) = \frac{{LI}_{end} - {LI}_{init}}{{t}_{G 2 S} - {t}_{LI , end}} ( t - {t}_{G 2 S , start} ) + {LI}_{init} .$$

In one form, the control system 200 may monitor the CCT of the lighting devices 106 in the habitable space 100 via a light sensor 134 and gradually reduce the CCT during the go-to-sleep scene by sending one or more control signals to the lighting devices 106. The CCT of a light source is a specification of the color appearance of the light emitted by a lamp, relating its color to the color of light from a reference source when heated to a particular temperature, measured in degrees Kelvin (K). Generally, a lower CCT has less impact on suppressing melatonin production than a higher CCT with more blue-spectrum content. In the default form, the CCT may be reduced from about 2700K to about 2000K during the go-to-sleep scene and can be reduced at a variety of speeds or in different intervals selected by the user. Depending on the lighting devices 106 associated with the habitable space 100, the control system 200 may adjust the CCT in a linear manner, or in some forms, can adjust the lighting intensity according to other non-linear functions.

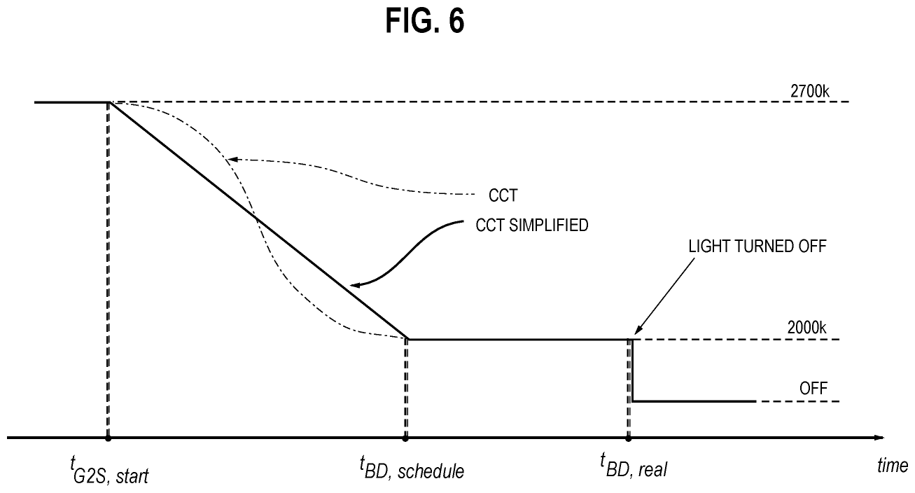

Similar to FIG. 5, FIG. 6 is an example graph showing the change in CCT of the lighting devices 106 by the control system 200 according to both a sigmoid transition and a linear transition. As shown, the CCT is reduced from its initial setting (for example, a default may be set to about 2700K) to its CCT at the end of the go-to-sleep scene (for example, a default may be set to about 2000K). As shown, the adjustment would occur at a specified rate between initiation of the go-to-sleep scene and the scheduled or predicted bedtime of the user. In the illustrated form, the following sigmoid function may be used to gradually reduce CCT in the habitable space as shown in accordance with FIG. 6.

$$CCT ( t ) = \frac{1}{1 + {e}^{k ( \frac{t - {t}_{G 2 S , start}}{{t}_{G 2 S} - {t}_{CCT , end}} - \frac{1}{2} )}} ( {CCT}_{init} - {CCT}_{end} ) + {CCT}_{end}$$

In the above function, t is the time during the go-to-sleep scene, t_{G2S,start} is the time the go-to-sleep scene is initiated by the control system, t_{G2S} is the amount of time the go-to-sleep scene should start before the bedtime of the user (e.g., a default of 30 minutes), CCT_{end} is the CCT at the end stage of the go-to-sleep scene, which is also the CCT at the bed time, and CCTᵢₙᵢₜ is the CCT at the beginning of the scene. These values can be set by the user in the basic settings. Generally, if CCT_{end} is lower than CCTᵢₙᵢₜ, CCT_{end} will be set as CCTᵢₙᵢₜ. Additionally, t_{CCT,end} is typically the duration the final CCT will last. By one approach, the default value of t_{CCT,end} is (t_{G2S}−20) min., which typically provides enough time for CCT transition (20 mins). Further, k is the steepness of the sigmoid function, which also impacts the smoothness at the beginning and ending of the CCT transition. In some configurations, the default value of k is 7, which can be updated after user feedback or otherwise modified by the user. Such values can also be updated based on the detected indicia in the habitable space 100 by the one or more sensors 104 as described above.

The example functions for reducing light intensity and CCT may be similarly employed for the reduction of temperature, reduction of speaker intensity, and adjustment of other devices 102 associated with the habitable space 100. In other forms, other functions may be used to smooth the transition between the awake state and the asleep state of the user.

Referring again to FIG. 4, at step 412, the go-to-sleep scene may end upon an end trigger (which may be a scheduled time) being detected or an end threshold being exceeded. In some forms, the go-to-sleep scene may be configured to last for a predetermined period of time (e.g., 30 minutes long, and starting 30 minutes before the desired bedtime of the user) such that the control system 200 will automatically end the scene once the period of time has elapsed. In other forms, end of the go-to-sleep scene may be based on one or more end triggers such that the scene may continue until the end trigger is detected. For example, upon detection of an end trigger the control system 200 may end the scene before the predetermined period of time has elapsed. Alternatively, the control system 200 may end the scene and additionally transition to a new scene, such as a stay asleep scene. Such a trigger condition could include completion of the predetermined time, detected biometric characteristics of the person, detected environmental characteristics of the habitable space 100, detected behavioral characteristics of the person, or a detected user input.

As described above, the end trigger may be based upon one or more biometric characteristics of the user detected by the one or more sensors 104. For example, the end of the go-to-sleep scene may be triggered by detection or upon determination that the user in the habitable space 100 has fallen asleep by one or more sensors 104 or wearable devices 128, detection of the user entering a specific sleep stage (e.g., REM 1, REM 2, slow-wave, etc.), detection that the user has been asleep for an extended period of time, detection of the user's heart rate being below a predetermined threshold for a period of time, detection that a person is dreaming, among others.

The end trigger may also be based upon one or more environmental characteristics of the habitable space 100 detected by the one or more sensors 104. For example, the end of the go-to-sleep scene may be triggered by a combination of two or more of detection via a sensor 104 that the user is in bed and has not moved for a period of time, detection that the noise level in the room has remained below a preset level for a period of time (e.g., via the microphone 126), detection of snoring, detection of the light level remaining low or off for a period of time (e.g., via the light detector 134), detection of white noise or other soothing sounds played by the user, detection of a temperature in the habitable space 100 being below a certain threshold (e.g., via the sensor 138), among others.

The end trigger may also be based upon one or more behavioral characteristics of the person or a user input received at the user interface 208 of the control system 200. For example, shortly after arriving home, the user may input in the user interface 208 that the user desires the go-to-sleep scene to end early that night.

Once the end trigger has been detected and the control system 200 ends the go-to-sleep scene, the control system 200 may update the scene settings based on indicia detected by the one or more sensors 104 during the scene and may additionally transition to another scene, such as a stay asleep scene. For example, if the control system 200 detects, or the user inputs, that the lighting devices 106 are too bright during the go-to-sleep scene and is reducing sleep quality, the control system 200 may be configured to adjust the overall light intensity or otherwise adjust the speed at which the light intensity is reduced during the scene.

During the scenes initiated or triggered by the control system 200 in habitable space 100, the control system 200 may be configured to store locally and/or remotely the data and indicia detected by the one or more sensors 104 and additionally store locally and/or remotely a log of events during the scene in the memory 206 thereof. Such event logs and data recording may be desirable for system diagnosis and error detection. In addition, the event logs and recorded data may be analyzed by the processor 202 such that the user's preferences and behavior patterns are detected and may be used to update the current algorithm executed by the processor 202 to adjust one or more of the scenes. The event log includes status of the system 200 when the system 200 has encountered an event, such as a change in system status. For example, the start trigger being detected for any given scene may be recorded in the event log for later analysis or diagnosis. The event log may also include various interval logs storing periodic values of the variables in the system (e.g., the indicia detected by the one or more sensors 104). The interval logs may contain information such as the time, the current scene implemented by the control system 200, the value detected by all sensors 104, and the value detected of the devices 102 associated with the habitable space.

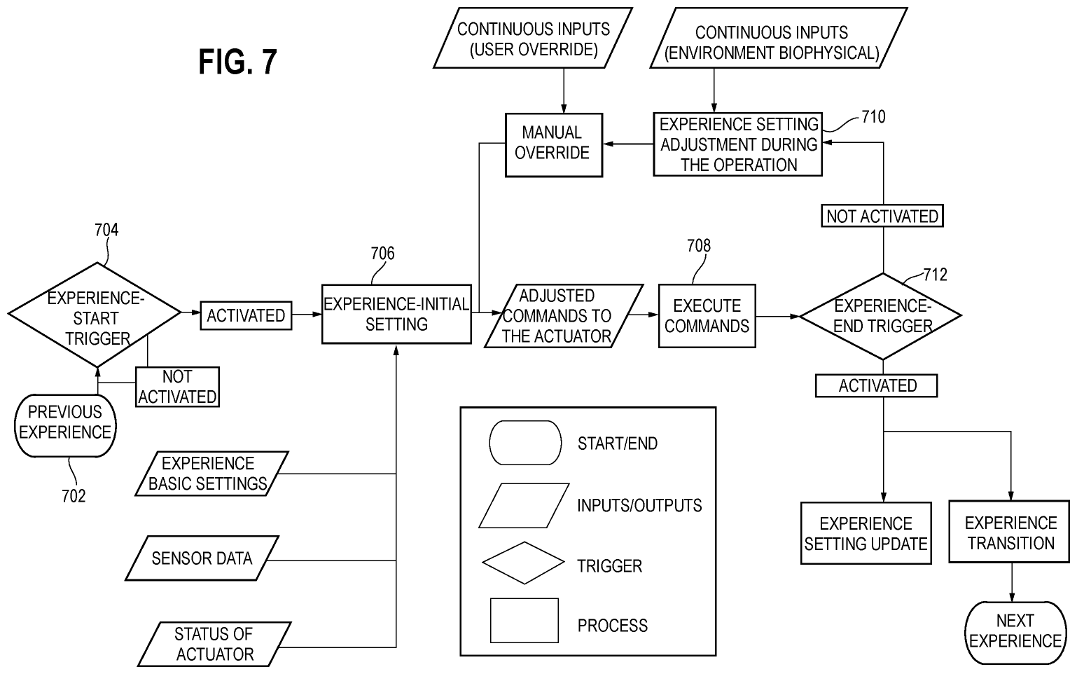

In other forms, the control system 200 may be configured to initiate or implement a scene to improve the user's sleep environment and hygiene during sleep. Specifically, FIG. 7 shows a logic flowchart that may be followed by the control system 200 while implementing the scene. Such a scene may be referred to hereinafter as a “stay asleep” scene. The stay asleep scene is configured to improve sleep hygiene of the user by maintaining the indoor environment and monitoring sleep quality while the user is sleeping. Similar to other scenes described herein, the user can specify settings for all of the devices in the habitable space through the user interface such that the control system may adjust operation of the devices or hold the devices at the desired settings while the user is sleeping.

As described, at step 702, the control system 200 may initiate the stay asleep scene upon termination of or transition from the go-to-sleep scene such that the user in the habitable space 100 is already asleep or should have already fallen asleep at a designated time. Alternatively, the control system 200 may transition to the stay asleep scene from another scene in the habitable space 100. In other forms, the control system 200 may be configured to initiate or trigger the stay asleep scene based at least in part on a start trigger. The start trigger may be based at least in part on indicia detected by the one or more sensors 104 in the habitable space 100.

For example, the start trigger at step 704 may be based at least in part on upon one or more biometric characteristics of the user detected by the one or more sensors. For example, the start of the stay asleep scene may be triggered by detection that the user in the habitable space 100 has fallen asleep by one or more sensors or wearable devices (e.g., wearable 128), detection of the user entering a specific sleep stage (e.g., REM 1, REM 2, slow-wave, etc.), detection that the user has been asleep for an extended period of time, detection of the user's heart rate being below a predetermined threshold for a period of time, detection that a person is dreaming, among others.

The start trigger may also be based at least in part upon one or more environmental characteristics of the habitable space 100 detected by the one or more sensors 104. For example, the start of the stay asleep scene may be triggered by detection via a sensor 104 that the user is in bed and has not moved for a period of time, detection that the noise level in the room has remained below a preset level for a period of time, detection of snoring, detection of the light level remaining low or off for a period of time, detection that a pet in the habitable space has fallen asleep, detection of white noise or other soothing sounds played by the user, detection of a temperature in the habitable space being below a certain threshold, among others.

The start trigger may also be based at least in part upon one or more behavioral characteristics of the person or a user input received at the user interface 208 of the control system 200. For example, shortly after arriving home, the user may input in the user interface 208 that the user desires the stay asleep scene to begin at a certain time during the night.

Once the trigger condition is satisfied, at step 708, the control system 200 begins to initiate the stay asleep scene by communicating signals to the devices 102 associated with the habitable space 100 based on the basic or initial settings (i.e., from setting up the system 200), indicia detected by the one or more sensors 104, and the status of the devices 102. For example, the temperature and humidity of the habitable space 100 may be adjusted by the HVAC system 118. The basic settings for the stay asleep scene may be default or scheduled settings, or may be input by the user at the user interface 208 during an initial installation or registration to reflect preferences of the user. For example, the user may input desired operation of the devices 102 in the habitable space 100 as described below.

After the control system 200 has caused initiation of the stay asleep scene and the basic settings for the devices 102 associated with the habitable space 100 have been implemented via the signals from the communication circuitry 204, at step 710, the control system 200 may be configured to continue measuring various indicia in the habitable space 100 via the one or more sensors 104 to adjust the scene over time.

Referring again to FIG. 7, at step 712, the stay asleep scene may end upon an end trigger being detected or an end threshold being exceeded. In some forms, the end of the stay asleep scene may be based on one or more end triggers such that the scene may continue until the end trigger is detected. Alternatively, the control system 200 may end the scene and additionally transition to a new scene, such as a wake-up scene. Such a trigger condition could include completion of a predetermined time, detected biometric characteristics of the person, detected environmental characteristics of the habitable space 100, detected behavioral characteristics of the person, or a detected user input.

Once the end trigger has been detected and the control system 200 ends the stay asleep scene, the control system 200 may update the scene settings based on indicia detected by the one or more sensors 104 during the scene and may additionally transition to another scene, such as a wake-up scene.

Similar to the go-to-sleep scene, the control system 200 is configured to store the data and indicia detected by the one or more sensors 104 and additionally store a log of events during the scene in the memory 206 thereof. The event log may also include various interval logs storing periodic values of the variables in the system (e.g., the indicia detected by the one or more sensors 104). The interval logs may contain information such as the time, the current scene implemented by the control system 200, the values detected by the sensors 104, and the values detected of the devices 102 associated with the habitable space 100. Based on the data detected by the sensors 104 (e.g., the various biometric sensors such as actigraphy based sensors and BCG sensors) while the user is asleep, the control system 200 may determine the sleep stages undergone for later analysis to determine sleep quality.

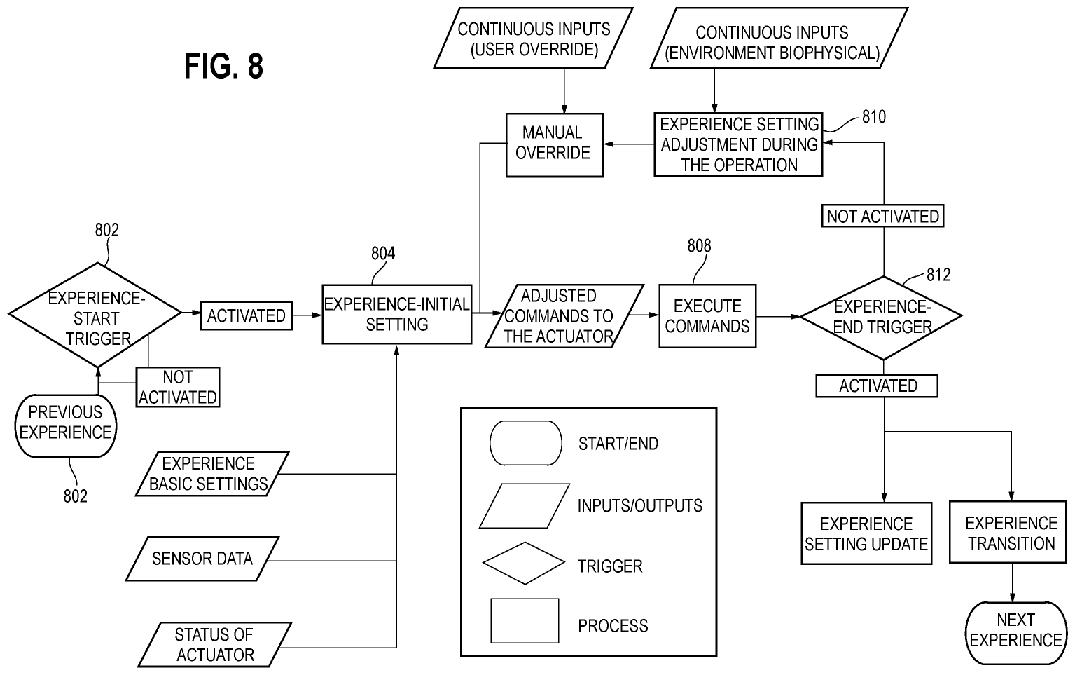

In other forms, the control system 200 may be configured to implement or transition to a scene to improve the user's sleep environment and hygiene while waking up from a sleep state. Specifically, FIG. 8 shows a logic flowchart that may be followed by the control system 200 while implementing the scene. Such a scene may be referred to hereinafter as a “wake-up” scene. The wake-up scene is configured to create a more pleasant experience for the user while waking up by, for example, gradually increasing light intensity, increasing temperature in the habitable space 100, opening window coverings 114, playing specific sounds, etc. Scientific literature suggests that adjusting certain environmental parameters during the wake-up time may provide greater alertness, better mood, lower sleep inertia, and improved circadian entrainment.

In one example wake-up scene, the lighting devices 106 may be programmed to provide a gradual increase in CCT (e.g., from an initial value of about 1400K to a final value of about 4000K) and intensity (e.g., from an initial value of about 10% to a final value of about 100%) over the course of 30 minutes prior to the scheduled wake-up time of the user. Over the last 2 minutes, the control system 200 may cause the speaker device 124 to play nature sounds (e.g., forest birds, ocean waves, rain, etc.) at a gradually increasing volume (e.g., from 30 dB to 55 dB), reaching a maximum dB level at the same time the final light intensity has been reached. In addition, the HVAC system 118 may be programmed to gradually increase the temperature by 2° C. over the course of the 30 minutes. At the end of the 30 minutes, the window coverings 114 may be moved to uncover the windows 112 to allow natural light in the habitable space. In some forms, the control system 200 may verify a sunrise time for the local time zone of the user, and cause the window coverings 114 to open if the sunrise occurs prior to the designated wake up time; otherwise, the control system 200 may inhibit opening of the window coverings 114 if the user is still sleeping due to privacy concerns. After the wake-up scene is complete (e.g., via the end trigger), the control system 200 may cause the light to slowly fade over a particular amount of time to correct the circadian setting for the particular time of day.

As described, the control system 200 may initiate the wake-up scene at a predetermined time before the user's desired wake up time such that the user may be gradually transitioned from a sleeping state to an awake state. For example, the scene may be initiated based on a preset alarm clock, a preset time on a calendar for waking up, among others. In some forms, the control system 200 may initiate the wake-up scene about 15 minutes before a desired wake-up time of the user, about 30 minutes before a desired wake-up time of the user, about 45 minutes before a wake-up time of the user, among others. So configured, during the time period in which the wake-up scene is implemented, there may be a gradual transition to an awake state of the user to improve user's waking up experience.

In other forms, the control system 200 is configured to initiate the wake-up scene based upon a predetermined trigger condition being met. Such a trigger condition could include completion of a previous scene (e.g., completion of a stay asleep scene), detected biometric characteristics of the person, detected environmental characteristics of the habitable space, detected behavioral characteristics of the person, or a detected user input.

In some forms, as shown at step 802, the control system may be configured to initiate the wake-up scene upon completion or near the completion of another scene within the habitable space 100, such as the stay asleep scene. For example, the control system 200 may transition between from the stay asleep scene to the wake-up scene automatically upon the stay asleep scene ending.

At step 804, the control system 200 is configured to initiate the wake-up scene based upon a predetermined schedule or other trigger condition being met. The start trigger may be based at least in part upon the biometric characteristics of the user detected by the one or more sensors 104. For example, the wake-up scene may be triggered by detection of the user in a lighter sleep stage (which may be more conducive to waking up), detection of the user's body temperature starting to rise, detection of the user having a full bladder, detection of stress, detection of a person being asleep for a minimum period of time, among others.

Additionally or alternatively, the start trigger may be based at least in part upon one or more environmental characteristics of the habitable space 100 detected by the one or more sensors 104. For example, the wake-up scene may be triggered by detection of an alarm going off, detection of the user getting out of bed, detection of rolling or other movement in the bed, detection of an emergency in the habitable space, detection of a specific sound (e.g., a phone ringing, a baby crying, cooking sounds, fire alarm, a doorbell, etc.), detection of the user turning on the lights, detection of the user turning on a television set, detection of sunrise (or a certain time after sunrise), detection of a signal by the user (e.g., a voice command to start the scene), detection of movement in another subspace within the habitable space 100, detection of movement or noise from a pet, receipt of a text message or email, a door or window being opened, among others.

In order to avoid instances where the user is already awake and does not wish to experience the wake-up scene, the control system 200 may be configured to only trigger the wake-up scene within a predefined time period after the designated wake-up time. By way of example, a threshold of 30 minutes may be set such that if the user inputs a wake-up time of 8:00 AM, and the control system 200 detects the user is awake at 8:45 AM based on the environmental characteristics, triggering of the wake-up scene may be inhibited as the wake-up scene may be unnecessary.

The start trigger may also be based at least in part upon the behavioral characteristics of the user either detected by the one or more sensors 104 or reported via the user interface 208 by the person. For example, the wake-up scene may be triggered by the user manually initiating the wake-up scene, input that a user needs to be someone at a specific time in the morning (e.g., pick up child, appointment, etc.), detection that the person went to sleep later than expected, among others.

Various other triggers, such as data from an external source, may likewise be utilized in connection with the control system 200 to determine when to initiate the wake-up scene. For example, the wake-up scene may be triggered by instruction from a third party, user input from a family member indicating the user should wake up, among others. In addition, the control system 200 may be configured to adjust the start trigger depending on changes in time zones (e.g., to accommodate for jet lag), and also changes in daylight savings time.

In some forms, combinations of detected triggers may be analyzed to determine whether to initiate the wake-up scene. For example, initiation of the wake-up scene may require multiple trigger conditions being met, such as both a detection that the user has gotten out of bed in connection with a detection of the lights being turned on. So configured, the control system 200 can increase the confidence interval that a user in the habitable space 100 is actually waking up.

In still other forms, the control system may be configured to monitor the user via the sensors over an extended period of time such that the control system may be able to predict a user's wake-up time based on historical data stored in a memory thereof and may be configured to implement the wake-up scene at a time based on the prediction. For example, if the control system detects that the user wakes up every morning at 6:30 AM, the control system may automatically initiate the wake-up scene between around 6:00 AM and 6:15 AM. Such a prediction may be based on, for example, an average wake time of the user over a prior 30-day period.

In addition, various other triggers or trigger conditions may be configured to cause a delay of the onset of the wake-up scene upon detection by the control system 200. The user may not always desire or schedule to wake up at the same time every morning, and the control system 200 may be able to adjust the wake-up scene based at least in part on indicia detected by the one or more sensors 104. For example, the wake-up scene may be delayed upon a detection of the user making a statement, gesture, or indication of desire to delay waking up, detection that the person is still in deep sleep, detection that an alarm has been turned off or delayed, detection that an activity previously scheduled that would cause the person to get up is now cancelled, detection of a preset wake-up time being changed, an indication from another person that the user does not need to wake up yet, determination that the user is on an off day from work or vacation day, detection that the person manually opted out of the scene, etc.

In some embodiments, the wake-up scene can also be manually disabled by the user through the user interface 208 associated with the control system 200. For example, the wake-up scene may be inhibited from triggering if the user is not detected in the habitable space (e.g., by the occupant-based sensors such as the camera 132 described above). Additionally, or alternatively, the wake-up scene may be inhibited from triggering if the control system 200 detects that the user is already awake. In such an instance, the control system 200 may be configured to instead initiate an alternative scene.

Once the trigger is satisfied or otherwise determined to have occurred, the control system 200 begins to initiate the wake-up scene by communicating signals to the devices 102 associated with the habitable space 100 based on the basic or initial default settings (i.e., from setting up the system), indicia detected by the one or more sensors 104, and the status of the devices 102. The basic settings for the wake-up scene may be default or scheduled settings or may be input or changed by the user at the user interface 208 during an initial installation or registration to reflect preferences of the user. For example, the user may input a typical wake time, a desired amount of sleep per night, and desired operation of the devices 102 in the habitable space 100 as described below. Alternatively, the control system 200 may predict or suggest a wake time and an amount of sleep per night based on the user's initial input.

The basic settings of the wake-up scene may include a desired light intensity during the wake-up scene such that the lighting devices 106 in the habitable space 100 may be adjusted by the control system 200 to the desired intensity either rapidly or gradually over a period of time. Once the wake-up scene is initiated, the lighting devices 106 may start gradually changing from either off or a low light intensity up to about 100% light intensity in the habitable space, which can be modified by the user.

The basic settings may also include a desired correlated color temperature during the wake-up scene such that the lighting devices 106 in the habitable space 100 may be adjusted by the control system 200 to a desired CCT either rapidly or gradually over a period of time. Once the wake-up scene is initiated, the lighting devices 106 will start changing from either off or a low CCT to about 4000K, which can be modified by the user.

In embodiments including a window shade or covering 114 that may be controlled via an actuator device 116, upon initiation of the wake-up scene, the control system 200 may cause the actuator device 116 to open in whole or in part the shade or covering 114 so as to increase the amount of natural light present in the habitable space 100. In some forms, the settings for the window shade or covering 114 may be binary (i.e., open or closed). In other forms, the settings for the window shade or covering 114 may provide a sliding scale such that the covering 114 may only be partially opened, or gradually opened, such that more natural light enters the habitable space 100 over time during the wake-up scene. As noted above, if the sunrise occurs prior to the wake time of the user, the window covering 114 may be opened at the wake up time. Otherwise, the control system 200 may inhibit the window covering 114 from opening if the user is still sleeping due to privacy concerns, to make it easier for the user to continue sleeping until the wake up time, or for other reasons.

The basic settings may also include a desired temperature setpoint for the habitable space 100 during the wake-up scene such that the HVAC system 118 or thermostat may be adjusted by the control system 200 to the desired temperature. In some forms, the desired temperature is the temperature adjusted to during the go-to-sleep scene such that the temperature in the habitable space 100 may gradually return to the temperature when the go-to-sleep scene was initiated the prior night. In other forms, the desired temperature increase during the wake-up scene may be set to a default of 2° C. and may be adjusted by the user in the range of 0-5° C. A user may adjust the temperature outside of this range, but the control system 200 may notify the user accordingly regarding same. Once the wake-up scene is initiated, the control system 200 will cause the HVAC system 118 to start changing the temperature in the habitable space 100 to the desired temperature to warm the habitable space 100. For example, the control system 200 may cause the temperature to change from about 25° C. to about 27° C.

In embodiments where the habitable space 100 includes one or more speaker devices 124, the basic settings may also include a desired speaker intensity or intensity transition or sound content during the wake-up scene. Once the wake-up scene is initiated, the control system 200 will cause the speaker device 124 to start changing the speaker intensity in the habitable space 100 to the desired intensity. During initial setup, the user may also select the desired speaker content. In some forms, the one or more speaker devices 124 may be configured to play soothing sounds, such as white noise, that are conducive to improving the transition between a sleep state and an awake state. In other forms, the speaker device 124 may play nature sounds, songs selected by the user, among others. As noted above, the speaker device 124 may be coupled to a streaming service and if the content selected by the user is no longer available (e.g., file missing, license expired, content not available at current license level, etc.), the control system 200 may use a default sound and notify the user that the selected content is not available.

The basic settings may also include a desired brightness of electronic devices associated with the habitable space 100 during the wake-up scene such that the displays of such electronic devices in the habitable space 100 may be adjusted by the control system 200 to the desired brightness. The control system 200 may be configured to communicate signals to the electronic devices in the habitable space 100 to adjust brightness thereof during the wake-up scene.

The basic settings may be based on, for example, current scientific literature or sleep related research as described above. The communication circuitry 204 of the control system 200 may be configured to receive updates from a remote server computer or other source to adjust such stock settings over time. As noted above, users may modify these settings via the user interface 208 of the control system 200, such as through an application on their smart phone or other mobile communication device.

In one form, the control system 200 may monitor some or all of the light intensity in the habitable space 100 via light sensor 134 and gradually increase the light intensity during the wake-up scene by sending one or more control signals to the lighting devices 106. In the default form, the light intensity may be increased from about 10% to about 100% during the wake-up scene and can be increased at a variety of speeds or in different intervals selected by the user. Depending on the number, capabilities and types lighting devices 106 associated with the habitable space 100, and perhaps their location within the habitable space 100, the control system 200 may adjust the lighting intensity in a linear manner, or in some forms, can adjust the lighting intensity according to other non-linear functions. Different lights may be adjusted in different ways to create the desired light intensity within the habitable space 100.

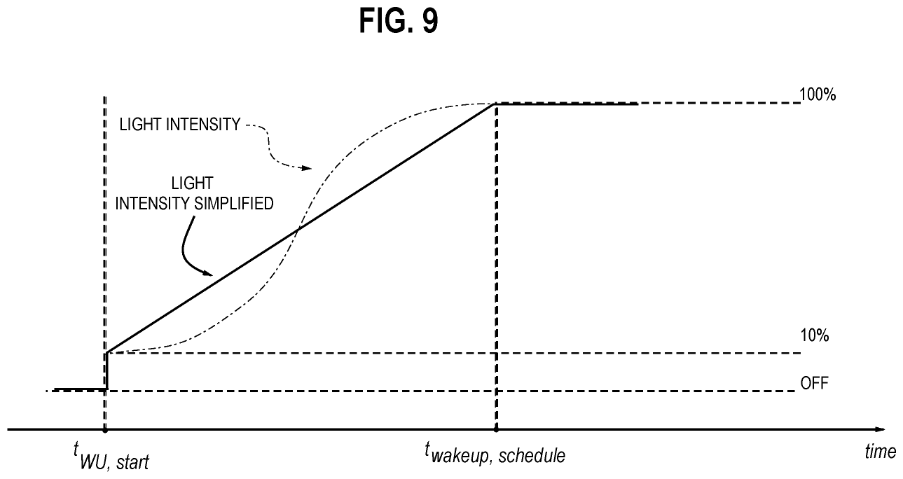

FIG. 9 is a an example graph showing the change in light intensity of the lighting devices 106 by the control system 200 according to both a sigmoid transition and a linear transition. As shown, the light intensity within the habitable space 100, or at least a portion of the habitable space 100, is increased from its initial intensity (for example, a default may be set to 10% light intensity) to its intensity at the end of the wake-up scene (for example, a default may be set to 100% light intensity). As shown, the adjustment would occur at a specified rate between initiation of the wake-up scene and the scheduled or predicted wake up time of the user. In the illustrated form, the following sigmoid function may be used to gradually increase the light intensity in the habitable space 100 as shown in accordance with FIG. 9.

$$LI ( t ) = \frac{1}{1 + {e}^{k ( \frac{t - {t}_{WU , start}}{{t}_{WU}} - \frac{1}{2} )}} ( {LI}_{init} - {LI}_{end} ) + {LI}_{end}$$

In the above function, t is the time during the wake-up scene, t_{WU,start} is the time the wake-up scene is initiated by the control system 200, t_{WU} is the amount of time the wake-up scene should start before the scheduled wake up time of the user (e.g., a default of 30 minutes), LI_{end} is the light intensity at the end stage of the wake-up scene, and LIᵢₙᵢₜ is the initial light intensity and the beginning of the wake-up scene (which may be equivalent to the light intensity at the end of the go-to-sleep scene and/or the light intensity during the stay asleep scene). These values can be set by the user in the scene basic settings. Additionally, t_{LI,end} is the duration the final light intensity will last. k is the steepness of the sigmoid function, which also impacts the smoothness at the beginning and ending of the light intensity transition. The default value of k is 7, which can be updated after user feedback or otherwise modified by the user. Such values can also be updated based on the detected indicia in the habitable space 100 by the one or more sensors 104 as described above.

If the sigmoid function identified above cannot be implemented for the lighting devices 106 in the habitable space 100 (e.g., there is no dimmer, or means for gradually increasing the light intensity over a period of time), a simplified linear change of light intensity may be implemented using the equation:

$$LI ( t ) = \frac{{LI}_{end} - {LI}_{init}}{{t}_{WU}} ( t - {t}_{WU , start} ) + {LI}_{init} .$$

In another form, the control system 200 may monitor the CCT of one or more of the lighting devices 106 in the habitable space 100 via light sensor 134 and gradually increase the CCT during the wake-up scene by sending one or more control signals to one or more of the lighting devices 106. In the default form, the CCT level in the habitable space, or a portion of the habitable space, may be increased from about 2000K to about 4000K during the wake-up scene and can be increased at a variety of speeds or in different intervals selected by the user. Depending on the number, capabilities and types of lighting devices 106 associated with the habitable space 100, and perhaps their location within the habitable space, the control system 200 may adjust the CCT in a linear manner, or in some forms, can adjust the lighting intensity according to other non-linear functions. Different lights may be adjusted in different ways to create the desired light intensity within the habitable space 100.

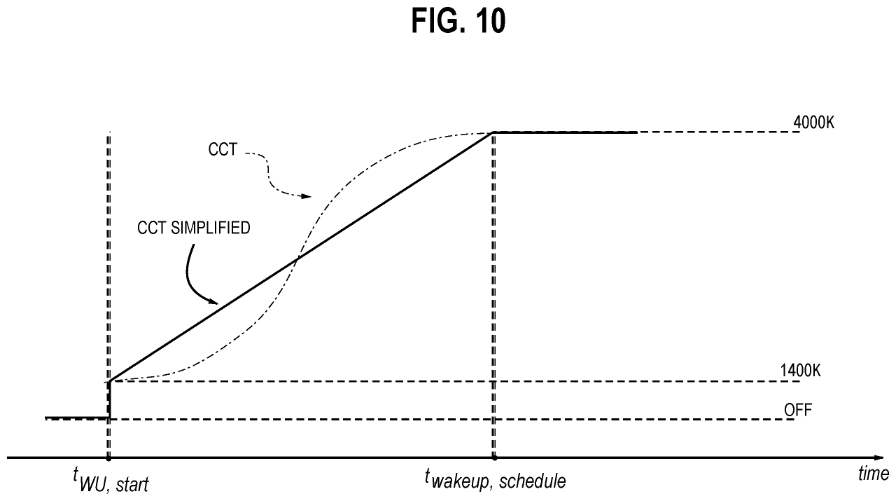

Similar to FIG. 9, FIG. 10 is a an example graph showing the change in CCT of the lighting devices by the control system 200 according to both a sigmoid transition and a linear transition. As shown, upon initiation of the wake-up scene in the habitable space 100, the CCT in the habitable space 100, or a portion of the habitable space 100, is increased from an initial CCT (for example, a default may be set to 0K) to a CCT of about 1400K, and may thereafter be gradually increased from about 1400K to a CCT at the end of the wake-up scene (for example, a default may be set to 4000K). As shown, the adjustment would occur at a specified rate between initiation of the wake-up scene and termination of the wake-up scene. In the illustrated form, the following sigmoid function may be used to gradually increase CCT in the habitable space 100 as shown in accordance with FIG. 10.

$$CCT ( t ) = \frac{1}{1 + {e}^{k ( \frac{t - {t}_{WU , start}}{{t}_{WU}} - \frac{1}{2} )}} ( {CCT}_{init} - {CCT}_{end} ) + {CCT}_{end}$$

In the above function, t is the time during the wake-up scene, t_{WU,start} is the time the wake-up scene is initiated by the control system 200, t_{WU} is the amount of time the wake-up scene should start before the wake time of the user (e.g., a default of 30 minutes), CCT_{end} is the CCT at the end stage of the wake-up scene, and CCTᵢₙᵢₜ is the CCT at the beginning of the scene (which may be equivalent to the CCT at the end of the go-to-sleep scene and/or the CCT during the stay asleep scene). These values can be set by the user in the basic settings. If CCT_{end} is lower than CCTᵢₙᵢₜ, CCT_{end} will be set as CCTᵢₙᵢₜ. k is the steepness of the sigmoid function, which also impacts the smoothness at the beginning and ending of the transition. The default value of k is 7, which can be updated after user feedback or otherwise modified by the user. Such values can also be updated based on the detected indicia in the habitable space 100 by the one or more sensors 104 as described above.

The example functions for increasing light intensity and CCT may be similarly employed for increasing temperature, increasing speaker intensity, and adjustment of one or more other devices 102 associated with the habitable space 100 or a portion of the habitable space 100. In other forms, other functions may be used to smooth the transition between the asleep state and the awake state of the user.

Referring again to FIG. 8, at step 812, the wake-up scene will end upon an end trigger being detected or an end threshold being exceeded. In some forms, the wake-up scene may be configured to last for a predetermined period of time (e.g., 30 minutes long, and starting 30 minutes before the desired wake-up time of the user) such that the control system 200 will automatically end the scene once the period of time has elapsed (e.g., once the user is awake) or at a scheduled time. In other forms, end of the wake-up scene may be based on one or more end triggers such that the scene may continue until the end trigger is detected. For example, upon detection of an end trigger the control system 200 may end the scene before the predetermined period of time has elapsed. Alternatively, the control system 200 may end the scene and additionally transition to a new scene. Such a trigger condition could include completion of the predetermined time, detected biometric characteristics of the user, detected environmental characteristics of the habitable space 100, detected movement or other behavioral characteristics of the user, detected user input, detected lack of occupancy in the space, etc.

As described above, the end trigger may be based upon one or more biometric characteristics of the user detected by the one or more sensors 104. For example, the end of the wake-up scene may be triggered by detection that the user has woken up, detection that the heart rate of the user is rising, detection that the user is no longer in a sleep stage, detection of movement (e.g., walking around the habitable space 100) above a defined threshold using a wearable, or detection of other characteristics or indicia indicating that the user is already awake.

The end trigger may also be based upon one or more environmental characteristics of the habitable space 100 detected by the one or more sensors 104. For example, the end of the go-to-sleep scene may be triggered by detection of excessive movement or that the user has gotten out of bed or left the habitable space 100, detection of the user initiating another scene, detection of a person staying out of bed for a designated period of time, detection of lights being turned on, detection of electronic devices being activated, detection of activity in another room (e.g., a child waking up), detection of a specific gesture or sound, detection of an emergency situation, detection of a change in schedule, etc.

The end trigger may also be based upon one or more behavioral characteristics of the user or a user input received at the user interface 208 of the control system 200. For example, the user may input in the user interface 208 that the user desires to opt out of the wake-up scene such that the wake-up scene is not triggered and the user can sleep for a longer duration of time.

Once the end trigger has been detected and the control system 200 ends the wake-up scene, the control system 200 may update or otherwise modify one or more of the scene settings based on indicia detected by the one or more sensors 104 and may additionally transition to another scene in the habitable space 100. For example, if the control system 200 detects, or the user inputs, that the lighting devices 106 are too bright during the wake-up scene and should be kept at a lower light intensity, the control system 200 may be configured to adjust the overall light intensity or otherwise adjust the speed at which the light intensity is reduced.

The control system 200 may be further configured to provide notifications in the form of educational tips or reminders to the user via the user interface 208 or another device in an effort to improve sleep quality. For instance, the communication circuitry 204 may communicate a notification signal to a user's mobile communication device to cause display of an educational tip thereon. As mentioned above, various behavioral factors can influence the sleep quality of the person. Specifically, behaviors such as caffeine consumption, times for eating meals, the quality of food eaten, physical activity, timing of sleep onset, and use of electronic devices before bed can all have adverse effects on the quality of sleep. Such notifications provided by the control system 200 may include information and tips about the effect of such behaviors on healthy sleep habits, and may be based at least in part on current scientific literature or sleep related research. Some illustrative examples of educational tips may include: (1) “To improve sleep quality, keep the bedroom quiet, dark, and at a cool temperature,” (2) “Getting at least 30 minutes of sunshine in the morning or early afternoon helps synchronize circadian rhythm,” (3) “Avoid napping in the late afternoon as it can negatively affect sleep quality,” (4) “Avoid caffeine before bedtime,” etc. In addition to educational tips being display on the screen of the user interface 208 such as the user's mobile phone, the control system 200 may be able to interact with a user via an audio alert or vibration.

The content of the educational tips or reminders may be selected by the processor 202 at least in part based on detected actions of the user within the habitable space 100. During operation, the control system 200 may detect actions of the user via the one or more sensors 104 and store such information in an event log in the memory 206. In turn, the processor 202 of the control system 200 may be configured to push selected educational tips to the user upon repeated detection or lack of detection of certain actions. For instance, it is known that increased physical activity can have a positive impact on sleep quality. The user may self-report any exercise via the user interface 208 of the control system 200 or such exercise data may be collected biometrically via a wearable device such that the control system 200 may provide notifications based on the user's detected physical activity. If the user indicates that the he or she has not exercised for a threshold duration of time (e.g., a few days, a week, etc.), the processor 202 of the control system 200 may be configured to cause the communication circuitry 204 to communicate a notification signal to the user interface 208 or a mobile communication device (e.g., smart phone 144) of the user to display an educational tip encouraging the user to exercise to improve sleep quality.

Additionally or alternatively, the control system 200 may assess sleep quality of the user and if the sleep quality is determined to be poor, the control system 200 may provide educational tips and reminders based at least in part on one or more characteristics of the environment that may be negatively affecting the user's sleep quality. For example, if one of the one or more sensors 104 detects that the user is initiating an activity that may have a negative impact on their sleep quality, they may receive an alert or other notification from the control system 200 notifying them of the potential negative effect and potentially prompting them to cease the activity. For example, if a noise sensor (e.g., microphone 126) detects sounds in the bedroom of the user over about 30dB shortly before the user's bedtime, the control system 200 may push a notification to the user informing the user to turn down any possible sources of noise in the bedroom.

In some embodiments, the control system 200 may communicate a notification signal for causing display of an educational tip or reminder within predetermined time windows or at scheduled times or during schedule time intervals. In some instances, certain behavior of the user that would negatively affect sleep quality if occurring shortly before bedtime would have no effect if occurring earlier in the day. For example, the control system 200 may be configured to only permit communication of certain notifications within, for example, up to 3 hours before the user's selected bedtime or only after the user is determined be in a specific habitable space (e.g., apartment, bedroom). As a result, if a noise sensor communicatively coupled to the control system 200 detects sounds over about 30 dB in the user's bedroom at 2:00 PM, the control system 200 may not communicate a notification signal to the user to suggest turning down sources of noise before bedtime. However, if such noise was detected an hour before the user's selected bedtime, the control system 200 would communicate a notification signal. In this manner, undesired notifications which may otherwise bother the user may be avoided.

In addition, the control system 200 may prompt the user via the user interface 208 occasionally to ask whether certain educational tips were helpful to the user. If the user indicates that the tips were not helpful, the control system 200 may be configured to inhibit communication of those or similar educational tips in the future.

The control system 200 may also provide one or more notifications in the form of reminders to the user during a scene, or at a time before the scene has initiated, to remind the user of various aspects of the scene (e.g., the control system 200 may remind the user during a go-to-sleep scene that bedtime is in 30 minutes, 15 minutes, etc.). The control system 200 also might be configured so that it does not repeat all or part of a notification within a designated time frame, if at all, or only have the occurrence of another specific amount of time or trigger (which might be a different trigger than what caused the control system 200 to send the notification the first time).

In some forms, the user may program or adjust the control system 200 to only provide notifications at certain frequencies or at certain times throughout the day. For example, the user may input in the user interface 208 that the user desires to receive notifications from the control system 200 at low, medium, or high frequencies. In other forms, the user can disable the notifications and reminders via the user interface 208 of the control system 200 such that no notifications are provided. In still other forms, the control system 200 may only provide notifications to the user upon the user interacting with the user interface 208. For example, the control system 200 may be configured such that the user may only receive notifications upon interacting with the user interface 208 and requesting an educational tip or report on the user's prior night's sleep activity.

The control system 200 may additionally be configured to send an aggregated analysis or report of the user's sleep quality at predetermined intervals. By one approach, the control system 200 may aggregate sleep quality information of the user biweekly (i.e., every two weeks) and communicate a compiled analysis to the user's mobile communication device. The aggregated analysis may be in the form of a chart or graph to illustrate certain aspects of the user's sleep quality. Based at least in part on the aggregated analysis, the control system 200 may then recommend helpful educational tips to improve factors that may be causing poor sleep quality. For example, if the control system 200 detects that the user hasn't fallen asleep at their designated bedtime for a threshold number of days during a two week interval, the aggregated analysis may include an educational tip concerning the importance of getting a proper amount of sleep each night.

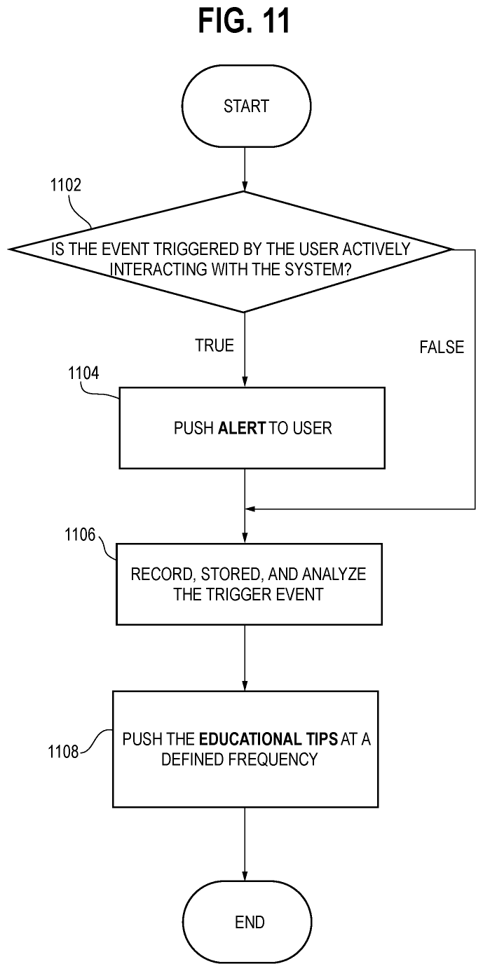

A process for providing educational tips or other notification to a user regarding the user's sleep hygiene is shown in more detail in FIG. 11. As described above, the educational tips may encourage users to power down devices at a specified interval before a selected bedtime, recommend exercise, etc., and may be based at least in part on current scientific literature or sleep related research. At step 1102 the processor 202 of the control system 200 determines whether an event triggered by the user is a result of the user interacting with the system (e.g., interaction with the user interface 208 thereof). If true, at step 1104, the processor 202 may cause the communication circuitry 204 to push an alert to the user by communicating a notification signal to the user's mobile communication device. At step 1106, the control system 200 may record and analyze the trigger event (i.e., what caused the control system 200 to cause display of the notification), and store the trigger event in an event log in the memory 206 of the control system 200. Thereafter, at step 1108, the control system 200 may push educational tips to the user at a desired frequency (e.g., as described above, the frequency may be set at differing intervals based on user input).

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system configured to initiate one or more scenes in a habitable space for a user, wherein the habitable space includes one or more devices, the system comprising:
a computing device including a processor, communication circuitry, and a memory; and
one or more sensors communicatively coupled to the computing device, the one or more sensors configured to detect indicia;
wherein the computing device is configured to trigger initiation of a sleep scene in the habitable space based at least in part on the detected indicia, and wherein the computing device is further configured to initiate the sleep scene by communicating, via the communication circuitry, a signal to at least one of the one or more devices in the habitable space, the signal configured to adjust operation of the at least one device.

2. The system of claim 1, wherein the detected indicia includes one or a combination of at least one biometric characteristic of a user, at least one environmental characteristic of the habitable space, and at least one behavioral characteristic of the user.

3. (canceled)

4. The system of claim 1, further comprising a user interface communicatively coupled to the processor, wherein the computing device is configured to trigger initiation of the sleep scene in the habitable space based at least in part on a user input, which includes one or a combination of:
one or more answers to a survey provided on the user interface;
a sleep schedule;
a sleep schedule change; and
a sleep goal;
wherein the computing device is further configured to initiate the sleep scene at a predetermined time before a sleep time of the user to automate sleep cues for the user in the habitable space.

5. The system of claim 1, wherein the computing device is inhibited from initiating the sleep scene when presence of the person in the habitable space is not detected by at least one of the one or more sensors in the habitable space.

6. The system of claim 1, wherein the computing device is further configured to trigger initiation of a wake-up scene in the habitable space based at least in part on the detected indicia, and wherein the computing device is further configured to initiate the wake-up scene by communicating, via the communication circuitry, a signal to at least one of the one or more devices in the habitable space, the signal configured to adjust operation of the at least one device.

7. (canceled)

8. (canceled)

9. The system of claim 1, wherein the computing device is configured to initiate at least one of:
a go-to-sleep scene followed by a stay asleep scene; or
a stay asleep scene followed by a wake-up scene.

10. The system of claim 4, wherein the computing device is further configured to analyze the user input and the detected indicia to determine adjustments to the sleep scene via at least one machine learning algorithm.

11. A method for increasing the likelihood that a person associated with a habitable space will fall asleep at a designated time, the method comprising:
sensing, via one or more sensors associated with the habitable space, one or more indicia, wherein the one or more sensors are communicatively coupled to a computing device;
triggering, via a processor of the computing device, a sleep scene for at least a portion of the habitable space upon a trigger being satisfied based at least in part on the detected indicia; and
implementing the sleep scene in at least the portion of the habitable space, wherein implementing includes communicating, via communication circuitry of the computing device, one or more signals to one or more devices positioned in the habitable space, the one or more signals configured to adjust operation of the one or more devices.

12. The method of claim 11, further comprising:
determining the trigger, wherein the trigger is indicative of a time of sleep of the person.

13. The method of claim 11, wherein the one or more sensors includes an environmental sensor, and wherein the method further comprises sensing one or more environmental parameters associated with the habitable space using the environmental sensor, and wherein the trigger being satisfied is further based at least in part on the one or more sensed environmental parameters.

14. The method of claim 11, wherein the step of implementing the sleep scene is configured to occur at a predetermined time period before a sleep time of the person.

15. The method of claim 11, further comprising:
adjusting the sleep scene, via the computing device, based at least in part on the indicia sensed by the one or more sensors.

16. The method of claim 11, wherein the step of implementing the sleep scene is inhibited when presence of the person in the habitable space is not detected by at least one of the one or more sensors.

17. The method of claim 11, further comprising:
triggering a wake scene based at least in part on the detected indicia.

18. The method of claim 17, further comprising:
implementing the wake scene in the habitable space, wherein implementing includes communicating, via communication circuitry of the computing device, one or more signals to one or more devices positioned in the habitable space, the signals configured to adjust operation of the one or more devices.

19-24. (canceled)

25. A method for increasing the likelihood that a person associated with a habitable space will wake up at a designated time, comprising:
sensing, by one or more sensors associated with the habitable space, one or more indicia, wherein the one or more sensors are communicatively coupled to a computing device;
triggering, by a processor of the computing device, a wake-up scene for at least a portion of the habitable space upon a trigger being satisfied based at least in part on the one or more sensed indicia; and
initiating the wake-up scene in the habitable space, wherein initiating includes communicating, via communication circuitry of the computing device, one or more signals to one or more devices positioned in the habitable space, the signals configured to adjust operation of the one or more devices.

26. The method of claim 25, further comprising:
determining the trigger, wherein the trigger is indicative of a wake time of the person.

27. The method of claim 25, wherein the one or more sensors includes an environmental sensor, and wherein the method further comprises sensing one or more environmental parameters associated with the habitable space using the environmental sensor; and
wherein the trigger being satisfied is further based at least in part on the one or more sensed environmental parameters.

28. (canceled)

29. The method of claim 25, wherein the step of initiating the wake scene is configured to occur at a predetermined time period before a wake time of the person.

30. The method of claim 25, further comprising:
adjusting the wake-up scene, via the computing device, based at least in part on the indicia sensed by the one or more sensors.

31-41. (canceled)
